SEQUENCE LISTING (2 sequences)

SEQ ID NO: 1 - protein, 348 aa, Escherichia coli
MEFFKKTALAALVMGFSGAALALPNITILATGGTIAGGGDSATKSNYTVGKVGVENLVNAVPQLKDIANVKGEQVVNIGSQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 2 - protein, 8 aa, synthetic construct
PHWSYLLR

(19) United States
(12) Patent Application Publication
(10) Pub. No.: US 2026/0048156 A1
(43) Pub. Date: Feb. 19, 2026

(54) FAP-TARGETING PHARMACEUTICAL PRODUCT FOR THERAPY AND DIAGNOSIS OF CANCERS

(71) Applicant: ATOMS FOR CURE GmbH, Berlin (DE)

(72) Inventors: Frank RÖSCH, Berlin (DE); Marcel MARTIN, Mainz (DE)

(73) Assignee: ATOMS FOR CURE GmbH, Berlin (DE)

(21) Appl. No.: 18/721,573

(22) PCT Filed: Dec. 20, 2022
(86) PCT No.: PCT/EP2022/087067

§ 371 Date: Jun. 18, 2024

(30) Foreign Application Priority Data
Dec. 20, 2021 (DE) 10 2021 133 942.1

(51) Int. Cl. A61K 51/04; A61K 47/55

(52) U.S. Cl. CPC A61K 51/0497; A61K 47/55; A61K 51/0446; A61K 51/0455; A61K 51/0459; A61K 2123/00

(57) ABSTRACT

An oncological, FAP-targeting pharmaceutical has the chemical structure: FAPi-R₁-FAPs-CT with R₁=formula (1) or formula (II) or formula (III), in which FAPi denotes an FAP inhibitor, FAPs denotes an FAP substrate, CT denotes a cytotoxin, MG denotes a labeling group for a radioisotope, TL denotes a tris linker, and L1, L2, L3, L4, L5, L6 denote bivalent linkers.

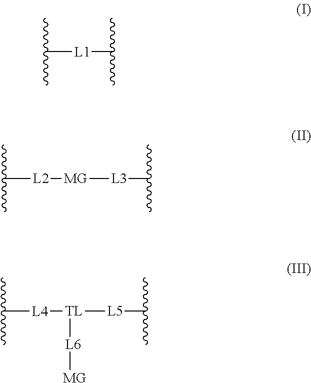

### SEQUENCE LISTING

This application contains a sequence listing whose file name is UMZ21025SequenceListing.txt, which was created on Oct. 20, 2025, has a size of 5 bites, and which is hereby incorporated by reference herein in its entirety.

### CROSS-REFERENCE TO RELATED APPLICATIONS

This application is being filed under 35 U.S.C. § 371 as a National Stage Application of pending International Application No. PCT/EP2022/087067 filed Dec. 20, 2022, which claims priority to the following parent application: German Patent Application No. 10 2021 133 942.1, filed Dec. 20, 2021. Both International Application No. PCT/EP2022/087067 and German Patent Application No. 10 2021 133 942.1 are hereby incorporated by reference herein in their entirety.

### FIELD OF THE INVENTION

The present invention relates to pharmaceutical compounds intended for therapy and diagnosis of cancers, which target the fibroblast activation protein (FAP).

The compounds of the invention have the structure

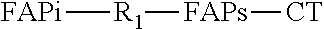

with

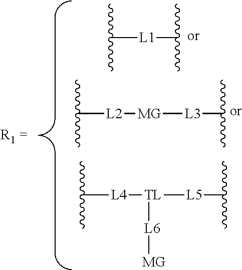

in which FAPi denotes an FAP inhibitor, FAPs denotes an FAP substrate, CT denotes a cytotoxin, MG denotes a labeling group for a radioisotope, TL denotes a tris linker, and L1, L2, L3, L4, L5, L6 denote bivalent linkers.

### BACKGROUND OF THE INVENTION

### Tumor Therapy and Diagnosis

Cytotoxic pharmaceuticals, for example doxorubicin, have been used for decades in chemotherapy. In conventional systemic chemotherapy, the cytotoxic pharmaceutical is administered intravenously, orally or peritoneally in relatively high dose. As well as cancer cells, cytotoxic pharmaceuticals also damage healthy tissue, especially cells with a high division rate, and cause severe, in some cases life-threatening, side effects that by no means rarely force stoppage of the treatment.

In order to alleviate side effects, low-dose targeted cytotoxic pharmaceuticals having high binding affinity for tumor cells have been used for some years. High tumor affinity is achieved by conjugating the cytotoxic active ingredient to a targeting vector. The targeting vector generally comprises agonists (substrates) or antagonists (inhibitors) of membrane-bound proteins that are greatly overexpressed on the wall of tumor cells by comparison with healthy cells. Targeting vectors may be simple organic compounds (small molecules), oligopeptides with natural or derivatized amino acids, and aptamers or monoclonal antibodies (mAb). The known compounds are configured for the specific targeting of particular tumor cells and have a tightly restricted spectrum of application.

The compounds of the invention are intended for cytotoxic and/or radiological therapy of various tumors and types of cancer, and for imaging diagnostics by positron emission tomography (PET) and single-photon emission computed tomography (SPECT).

The FAP inhibitor present in the compounds of the invention and the FAP substrate target the fibroblast activation protein (FAP). Since FAP is overexpressed in the tumor stroma of various tumors, the compounds of the invention are suitable for the treatment and diagnosis of numerous cancers.

In nuclear-medical diagnosis, tumor cells or metastases are visualized with the aid of a radioisotope, for example gallium-68 (⁶⁸Ga) or technetium-99m (^{99m}Tc). For metallic radioisotopes, preference is given to using coordinatively binding or complexing radiotracers. The radiotracers comprise, as an essential chemical component, a chelator for effective and stable complexation of the metallic radioisotope, and one or more targeting vectors that bind to biological target structures in the tumor tissue. In general, the targeting vector has a high affinity for trans-membrane receptors, proteins, enzymes or other structures of tumor cells. In addition, radiotracers containing nonmetallic radioisotopes such as fluorine-18 (¹⁸F) or iodine-123 (¹²³I) and iodine-131 (¹³¹I) are used. Nonmetallic radioisotopes are covalently bound in the respective radiotracer.

After intravenous injection into the bloodstream, the radiotracer accumulates on or in tumor cells or metastases. In order to minimize the radiation dose in healthy tissue, radioisotopes with a short half life of a few hours to a few days are used.

By contrast, in nuclear-medical therapy, elevated radiation doses are used in order to inflict maximum damage on carcinogenic lesions. For this purpose, for example, beta-minus-emitting radioisotopes are used, such as lutetium-177 (¹⁷⁷Lu), yttrium-90 (⁹⁰Y) and iodine-131 (¹³¹I), or alpha emitters such as actinium-225 (²²⁵Ac) or lead-212 (Pb²¹²). Alpha and beta-minus particles have a short range in tissue, an advantageous property for selective irradiation and damage of the tumor or metastases, but minimization of the radiation dose for the surrounding healthy tissue.

In the last few years, the combination of diagnosis and therapy, referred to among specialists as theranostics, has become increasingly important. The radiotracers that are used here for diagnostics and therapy—apart from the radioisotope—have a similar and preferably the same chemical structure. For PET diagnostics and radionuclide therapy, primarily gallium-68 and, respectively, lutetium-177 are used because both radioisotopes can be complexed with the DOTA chelator. This fact enables the use of the same labeling precursor for diagnosis with ⁶⁸Ga and radioendotherapy with ¹⁷⁷Lu, and hence direct translation from diagnostics to therapy.

In the known methods, it should always be remembered that a radiological labeling group, for example a chelator, affects the chemical and pharmacokinetic properties of a radiotracer. Accordingly, in the event of exchange of the labeling group of the radiotracers or labeling precursors modified in such a way, a reevaluation is necessary. This relates primarily to the biochemical and pharmacokinetic properties of the radiotracer and, to a certain degree, also to the coordination or conjugation of the respective radioisotope. The labeling group affects the biological and nuclear-medical potency of the respective labeling precursor to a crucial degree.

In addition to a high affinity, a radiotracer or labeling precursor must meet further demands, such as
rapid and effective complexation of the respective radioisotope;
high selectivity for tumor cells and metastases relative to healthy tissue;
in vivo stability, i.e. biochemical stability in blood serum under physiological conditions.

### Tumor Stroma

Malignant epithelial cells are a component of many tumors and tumor types and at a size of 1-2 mm at the latest form a tumor stroma surrounding the tumor.

The tumor stroma comprises various nonmalignant types of cells that are part of the tumor microenvironment surrounding the tumor cells. The tumor stroma may account for up to 90% of the total tumor mass. It plays an important role in the development, growth and metastasis of tumors, and in the supply of the tumor cells.

The most important components of the tumor stroma are the extracellular matrix including various cytokines, endothelial cells, pericytes, macrophage, immunoregulatory cells and activated fibroblasts. The activated fibroblasts surrounding the tumor are referred to as cancer-associated fibroblasts (CAFs).

In the course of tumor development, CAFs change their morphology and biological function. These changes are induced by intercellular communication between cancer cells and CAFs. In this context, CAFs form an environment that promotes the growth of the cancer cells. It has been found that therapies that target solely cancer cells are inadequate. Effective therapies must include the tumor microenvironment and hence also the CAFs.

In more than 90% of all human epithelial carcinomas, CAFs overexpress the fibroblast activation protein (FAP). Therefore, FAP represents a promising point of attack for nuclear-medical diagnosis and therapy. Suitable affine biological targeting vectors for FAP labeling precursors are FAP inhibitors (FAPI or FAPi). The role of FAP in vivo is not yet fully understood, but it is known that it is an enzyme having unique catalytic activity. It has both dipeptidyl-peptidase activity (DPP) and prolyloligopeptidase activity (PREP). Accordingly, useful inhibitors are those that inhibit the DPP activity and/or the PREP activity of FAP. The decisive factor is the selectivity of the respective inhibitor with respect to related enzymes, such as the dipeptidylpeptidases DPPII, DPPIV, DPP8 and DPP9, and homologous prolyloligopeptidases (PREP). In the case of cancer types where both FAP and PREP are overexpressed, it is also possible, however, to use inhibitors that do not have such high selectivity for PREP and FAP and inhibit both enzymes.

Since 2013, high-affinity and selective FAP inhibitors have been known, which consist of a modified glycine-proline unit and a quinoline group coupled thereto. Two such FAP inhibitors with the chemical structure (S)—N-(2-(2-cyanopyrrolidin-1-yl)-2-oxoethyl)quinoline-4-carboxamide and the difluorinated derivative thereof with the abbreviation “UAMC1110” are shown in scheme 1.

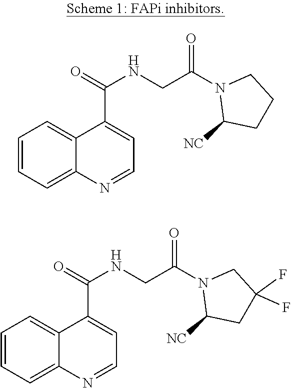 [SMILES: N#C[C@@H]1CCCN1C(=O)CNC(=O)c1ccnc2ccccc12 ; N#C[C@@H]1CC(F)(F)CN1C(=O)CNC(=O)c1ccnc2ccccc12]

The difluorinated FAP inhibitor UAMC1110 is used in a known labeling precursor for nuclear-medical applications, referred to as “FAPI-04” and shown in Scheme 2. As well as FAPI-04, scheme 2 shows three further known labeling precursors FAPI-21, FAPI-46 and DOTA.SA.FAPI. In the case of DOTA.SA.FAPi, the pharmacophoric FAPi unit is coupled to the DOTA chelator via a 4-aminobutoxy unit or a squaric acid-ethylenediamine unit.

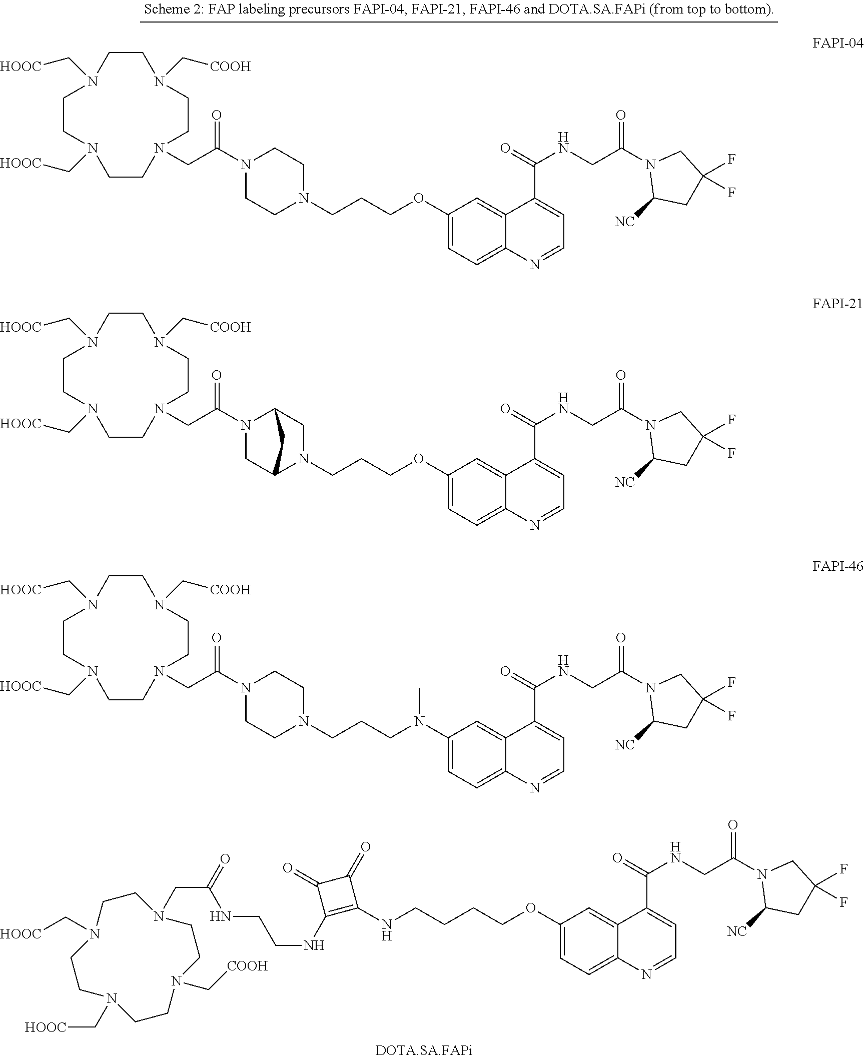

Scheme 3 shows newer dimeric labeling precursors, referred to as “DOTA-2P(FAPI)₂” and “DOTAGA.(SA.FAPi)₂”, each of which contains two structurally identical FAPi targeting vectors.

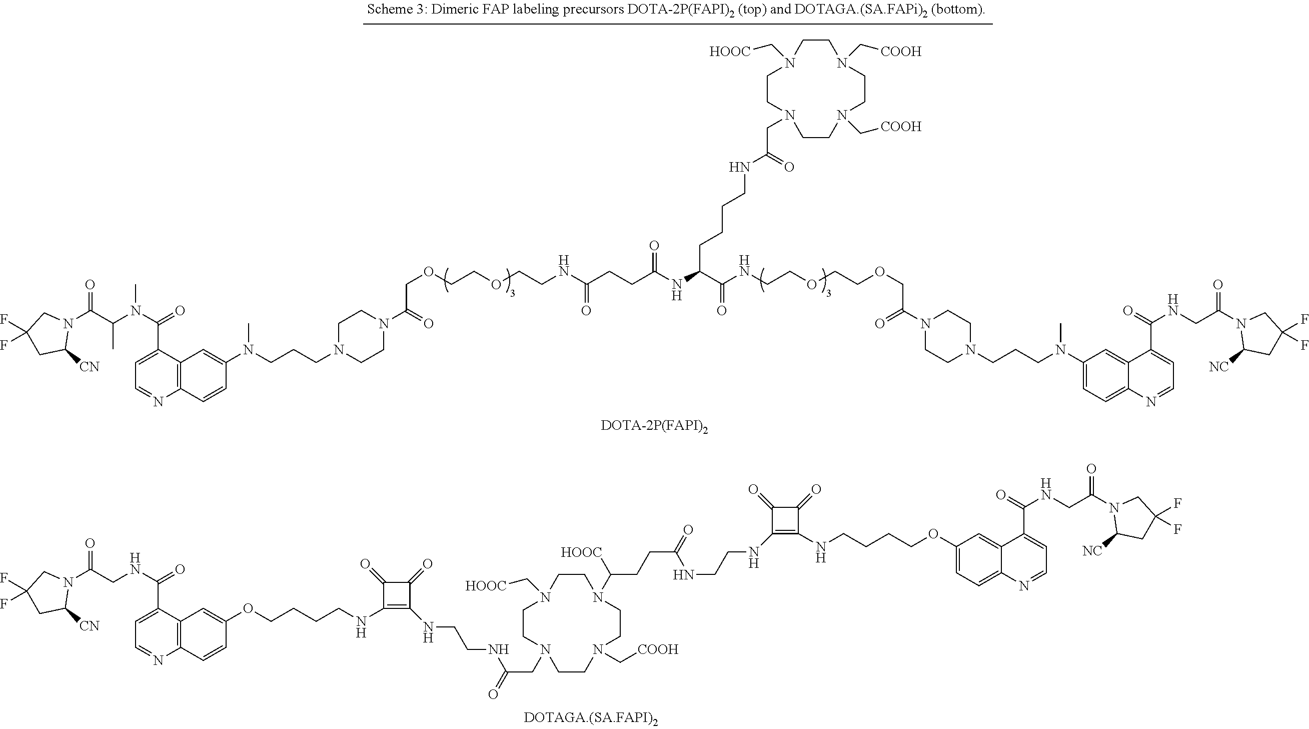

Radiotracers based on the labeling precursors shown in scheme 3 are notable for a considerably longer tumor residence time compared to those based on monomeric labeling precursors (cf. scheme 2).

Additionally known are highly selective FAP substrates as shown in scheme 4, which, in analogy to the aforementioned FAP inhibitors, include a 4,4′-difluorinated pyrrolidine ring. By contrast with the FAP inhibitors according to scheme 1, the nitrile group in the FAP substrates in scheme 4 is substituted by an amide bond. In the FAP substrates, various aromatic units, such as 7-amino-4-methylcoumarin (AMC) or p-nitroaniline (pNA), are conjugated via an amide bond. The substrates shown in scheme 4 are enzymatically cleaved by FAP, with cleavage of the glycine-proline unit of the substrates by the endopeptidase present in FAP. Otherwise, the substrates in scheme 4 are cleavable solely by a prolylendopeptidase (PREP) with appropriate enzymatic activity.

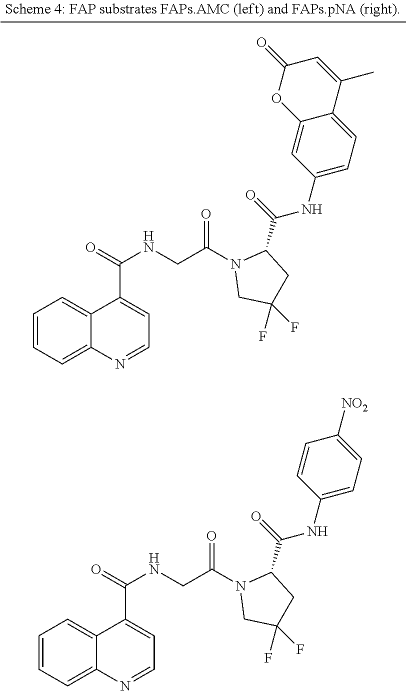 [SMILES: Cc1cc(=O)oc2cc(NC(=O)[C@@H]3CC(F)(F)CN3C(=O)CNC(=O)c3ccnc4ccccc34)ccc12 ; O=C(NCC(=O)N1CC(F)(F)C[C@H]1C(=O)Nc1ccc([N+](=O)[O-])cc1)c1ccnc2ccccc12]

The prior art discloses a multitude of labeling precursors for theranostics of cancers with radioisotopes.

WO 2019/154886 A1 discloses labeling precursors and radiotracers for the visualization of epithelial carcinomas and tumor stroma, including the compound FAPI-04 shown in scheme 2.

Also known are cytostatic drug delivery systems with low molecular weight targeting vectors for the targeting of FAP. In the prior art, for example, the cytostatic doxorubicin is linked via an amide bond to a glycine-proline unit. Scheme 5 shows two such compounds: FTPD (Z-Gly-Pro-doxorubicin) on the left and PhAc-ALGP-Doxo on the right, which are used as prodrugs. The prodrugs first have to be cleaved in order to release cytotoxically active doxorubicin.

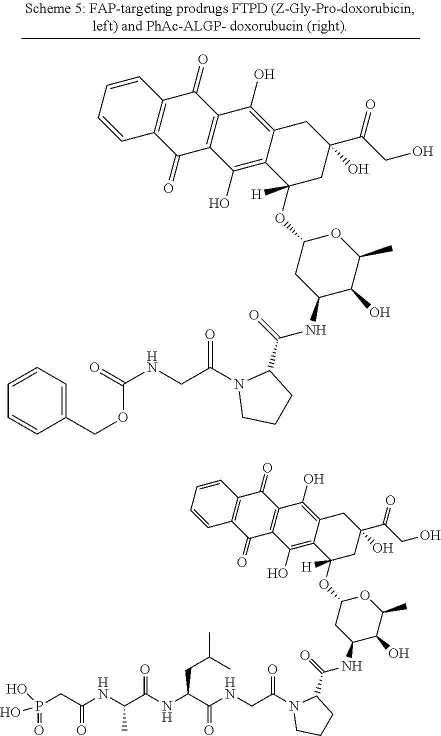

In in vivo studies on mice with prodrugs of such configuration give promising results.

### SUMMARY OF ADVANTAGEOUS EMBODIMENTS OF THE INVENTION

In spite of numerous advances in the diagnosis and treatment of cancer, there is still a need for oncological pharmaceuticals having improved
affinity and selectivity;
cytotoxicity;
pharmacokinetics;
elevated PET and SPECT image contrast or signal uptake value (SUV);
simple clinical use; and
extended patient population.

### DETAILED DESCRIPTION OF ADVANTAGEOUS EMBODIMENTS OF THE INVENTION

The object addressed by the invention is accordingly that of providing oncological pharmaceuticals having improved properties over known compounds. This object is achieved by a compound having the structure

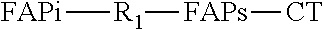

with

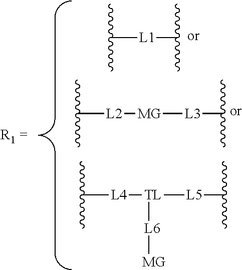

in which FAPi denotes an FAP inhibitor, FAPs denotes an FAP substrate, CT denotes a cytotoxin, MG denotes a labeling group for a radioisotope, TL denotes a tris linker, and L1, L2, L3, L4, L5, L6 denote bivalent linkers.

Advantageous embodiments of the compound of the invention are characterized by the following further features in any combination, provided that the combined features are not contradictory and according to which:
FAPs has one of the structures [1] to [20]:

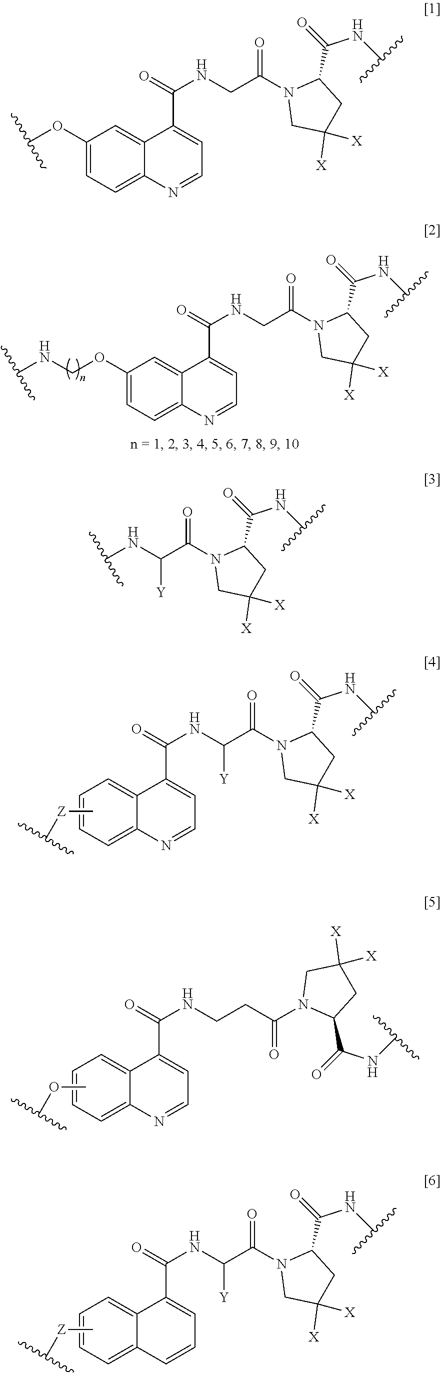

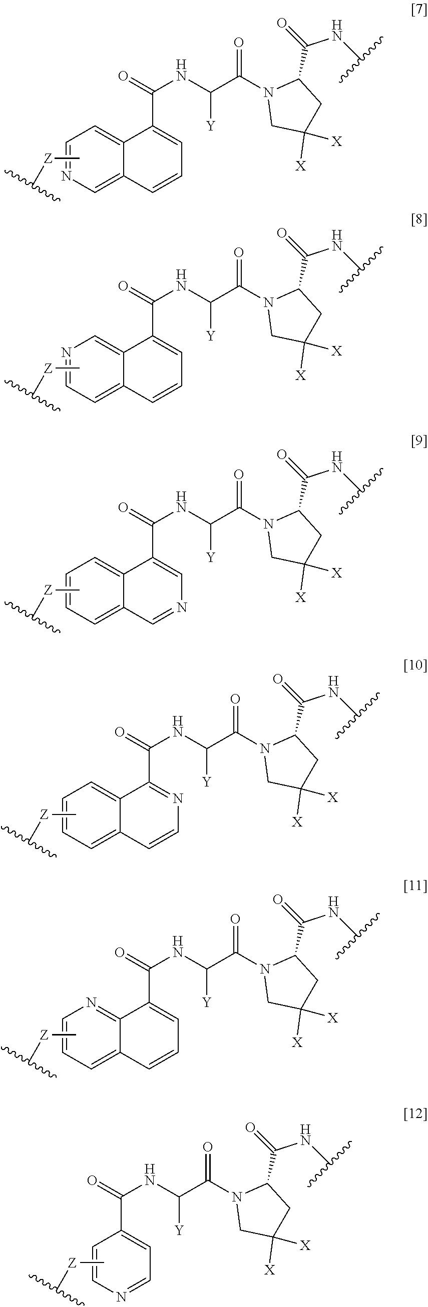

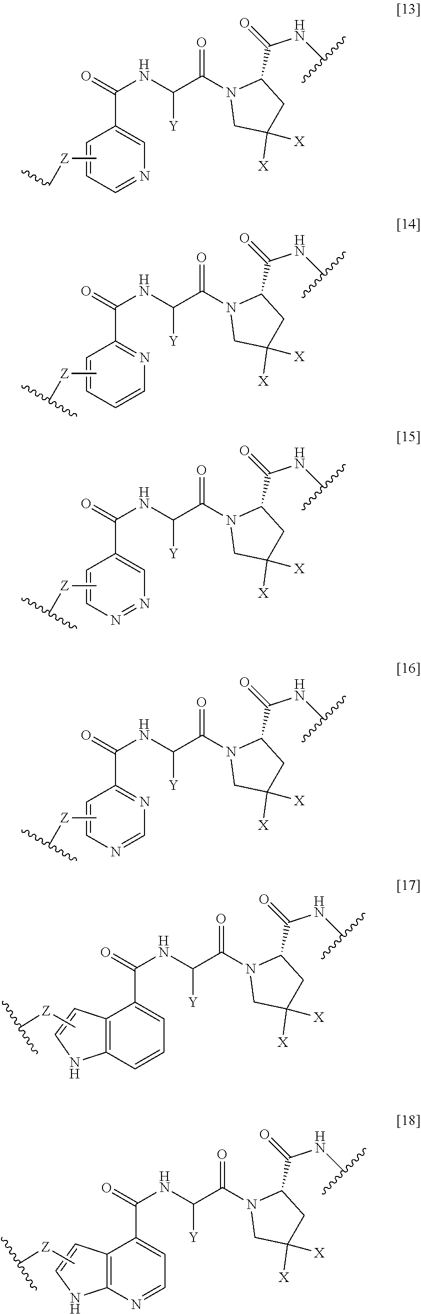

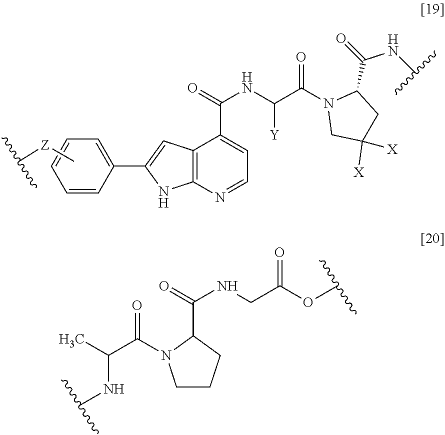

where [20] represents the amino acid sequence Ala-Pro-Gly;
where
X=H or F;
Y=H, CH₃, CH(CH₃)₂, C(CH₃)₃ or (CH₂)ₙCH₃ with n=1, 2, 3, 4, 5, 6, 7, 8, 9 or 10; and
Z is chosen from the group comprising alcohol, amidine, amine, amide, carboxamide, thioamide, imide, imide ester, imine, urea, thiourea, guanidine, carbonate, carboxylic ester, carbamate, ether, thioether, ester, ketone, phosphate, phosphonate, phosphinate, sulfonic ester, sulfinic ester, sulfone, thiol, disulfide, boronic ester, silyl ether radicals and derivatives thereof;
FAPi has one of the structures [21] to [46]:

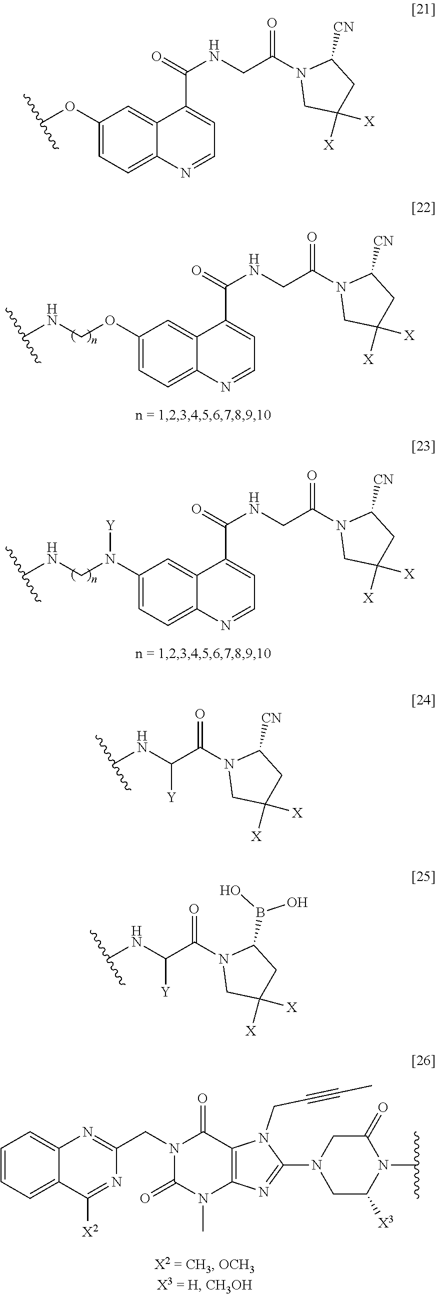

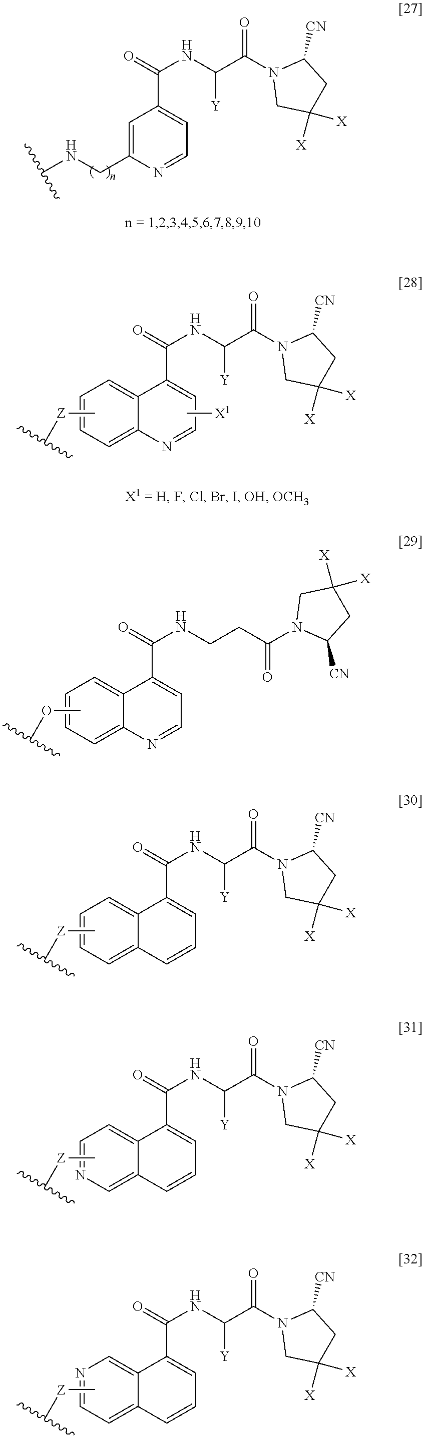

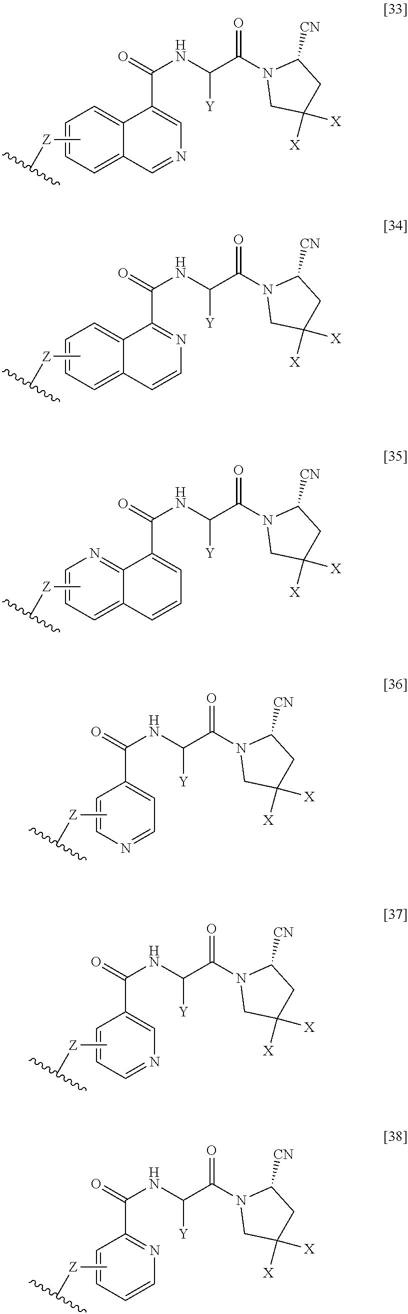

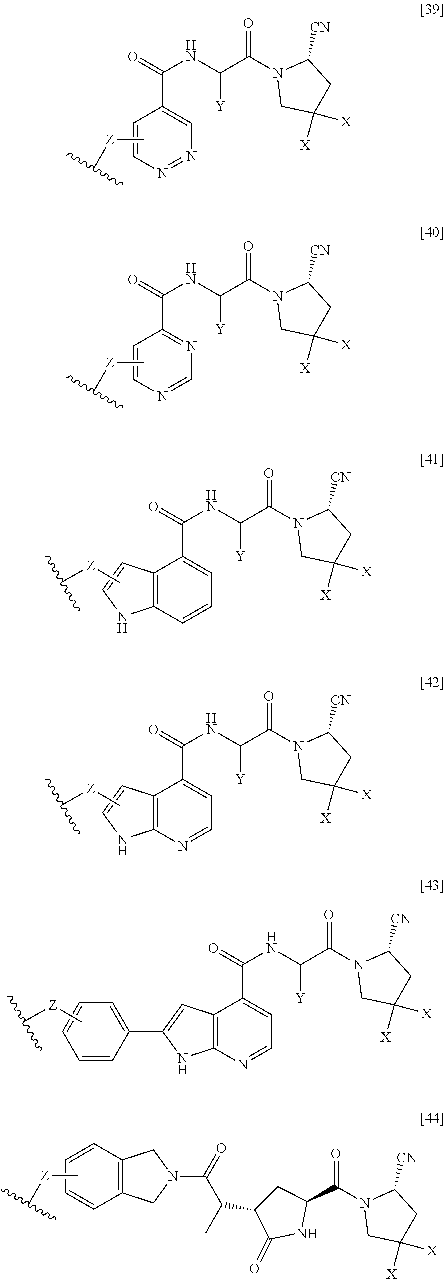

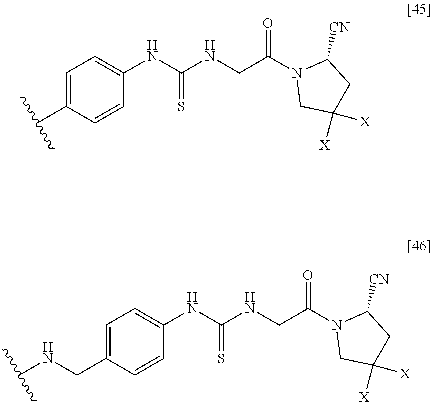

where
X=H or F;
Y=H, CH₃, CH(CH₃)₂, C(CH₃)₃ or (CH₂)ₙCH₃ with n=1, 2, 3, 4, 5, 6, 7, 8, 9 or 10;
Z is chosen from the group comprising alcohol, amidine, amine, amide, carboxamide, thioamide, imide, imide ester, imine, urea, thiourea, guanidine, carbonate, carboxylic ester, carbamate, ether, thioether, ester, ketone, phosphate, phosphonate, phosphinate, sulfonic ester, sulfinic ester, sulfone, thiol, disulfide, boronic ester, silyl ether radicals and derivatives thereof;
the linkers L1, L2, L3, L4, L5, L6 are independently chosen from the group comprising

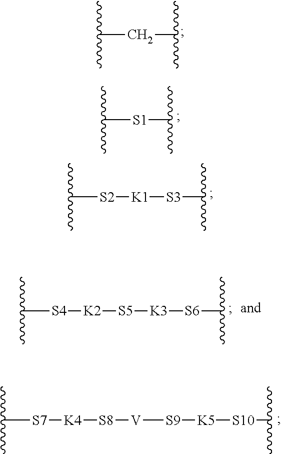

where the spacers S1, S2, S3, S4, S5, S6, S7, S8, S9, S10 are independently chosen from the group comprising a mono- or oligomer composed of m alkyl units, polyethylene glycol (—(CH₂CH₂O)ₙ—), polypropylene glycol (—(CH₂CH(CH₃)₂O)ₙ—), polyalkylene glycol ether (—((CH₂)ₙO)—), polyamine (—((CH₂)ₙNH)—), polyamide (—((CH₂)ₙCONH)—), polyester (—((CH₂)ₙCOO)—), polyurethane (—((CH₂)ₙNHCOO)—), polyurea (—((CH₂)ₙNHCONH)—) and peptides having m amino acids with m=1, 2, 3, 4, 5, 6, 7, 8, 9, 10, 11, 12, 13, 14, 15, 16, 17, 18, 19 or 20;
where each of the spacers S1, S2, S3, S4, S5, S6, S7, S8, S9, S10 is independently an oligopeptide having m amino acids chosen from the group comprising Ala, Arg, Asn, Asp, Cys, Gln, Glu, Gly, His, lie, Leu, Lys, Met, Phe, Pro, Ser, Thr, Trp, Tyr, Val, Pyl, Sec, GABA (γ-aminobutyric acid), homoserine, DOPA (3,4-dihydroxyphenylalanine), citrulline, β-alanine and thyroxine with m=1, 2, 3, 4, 5, 6, 7, 8, 9, 10, 11, 12, 13, 14, 15, 16, 17, 18, 19 or 20;
where the coupling groups K1, K2, K3, K4, K5 are independently chosen from the group comprising an alcohol, amidine, amine, amide, carboxamide, thioamide, imide, imide ester, imine, urea, thiourea, guanidine, carbonate, carboxylic ester, carbamate, ether, thioether, ester, ketone, phosphate, phosphonate, phosphinate, sulfonic ester, sulfinic ester, sulfone, thiol, disulfide, boronic ester, silyl ether radical and derivatives thereof; and
where the linkage unit V is chosen from
   one of the structures [47] to [50]:

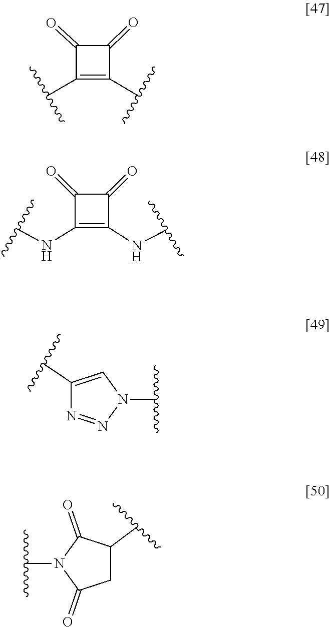 [SMILES: *c1c(*)c(=O)c1=O ; *Nc1c(N*)c(=O)c1=O ; *c1cn(*)nn1 ; *C1CC(=O)N(*)C1=O]

where [47] represents squaric acid, [48] squaric acid diamide, [49] a 1,4-disubstituted 1,2,3-triazole, and [50] a 1,3-disubstituted succinimidyl radical;
from the group comprising a residue of an amino acid such as Ala, Arg, Asn, Asp, Cys, Gln, Glu, Gly, His, lie, Leu, Lys, Met, Phe, Pro, Ser, Thr, Trp, Tyr, Val, Pyl, Sec, GABA (γ-aminobutyric acid), homoserine, DOPA (3,4-dihydroxy-phenylalanine), citrulline, β-alanine, thyroxine; or
from the group comprising a benzene, phenol, cyclopentane, cyclohexane, pyridine, pyridazine, pyrimidine, pyrazine, piperidine, piperazine, 1,2,3-triazine, 1,2,4-triazine, 1,3,5-triazine, 1,2,3,4-tetrazine, 1,2,3,5-tetrazine, 1,2,4,5-tetrazine, thiazine, oxazine, pyrrole, pyrrolidine, pyrazole, imidazole, tetrahydroimidazole, 1,2,4-triazole, tetrazole, thiophene, furan, 1,2-thiazole, 1,3-thiazole, thiadiazole, 1,2-oxazole, 1,3-oxazole, 1,2,3-oxadiazole, 1,2,4-oxadiazole, 1,2,4-oxadiazole, 1,2,5-oxadiazole, naphthalene, indene, indole, isoindole, indazole, quinoline, isoquinoline, quinazoline, quinoxaline, cinnoline, phthalazine, tetrahydroquinoline, tetrahydroisoquinoline, quinolizidine, indolizidine, 2H-chromene, 4H-chromene, 2H-thiochromene, 4H-thiochromene, coumarin, purine radical and derivatives thereof;
the tris linker TL linker is chosen from one of the structures [51] to [115]:

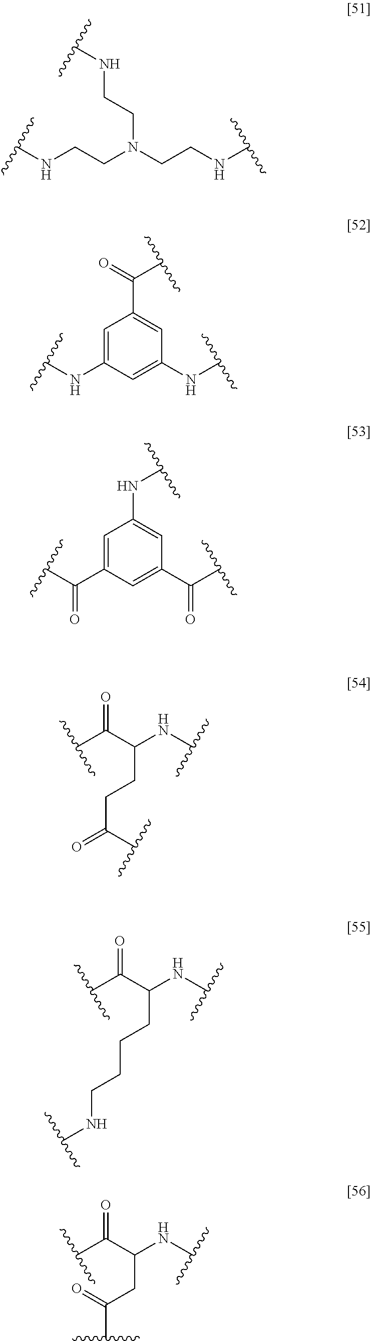 [SMILES: *NCCN(CCN*)CCN* ; *Nc1cc(N*)cc(C(*)=O)c1 ; *Nc1cc(C(*)=O)cc(C(*)=O)c1 ; *NC(CCC(*)=O)C(*)=O ; *NCCCCC(N*)C(*)=O ; *NC(CC(*)=O)C(*)=O]

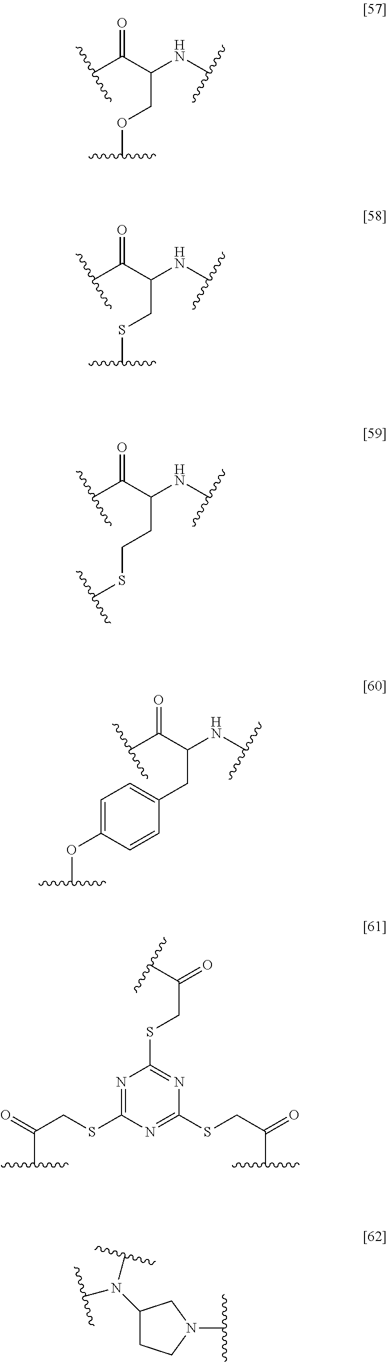 [SMILES: *NC(CO*)C(*)=O ; *NC(CS*)C(*)=O ; *NC(CCS*)C(*)=O ; *NC(Cc1ccc(O*)cc1)C(*)=O ; *C(=O)CSc1nc(SCC(*)=O)nc(SCC(*)=O)n1 ; *N1CCC(N(*)*)C1]

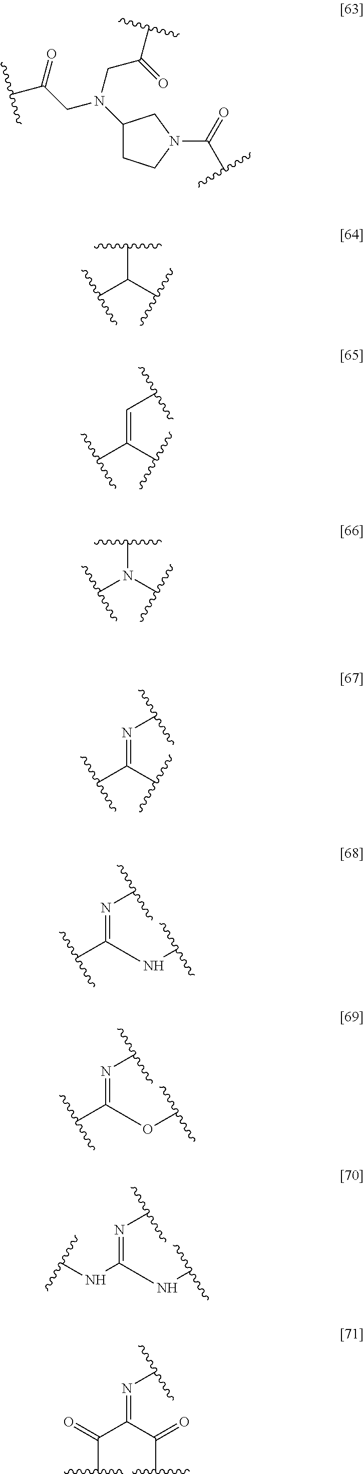

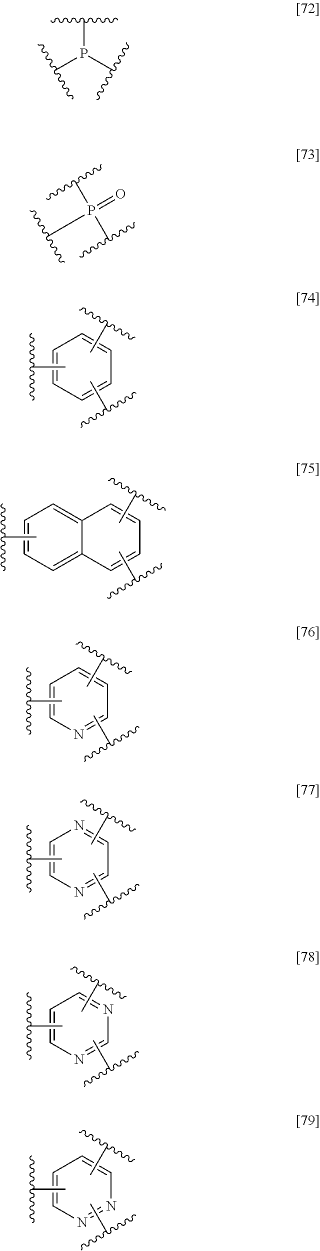

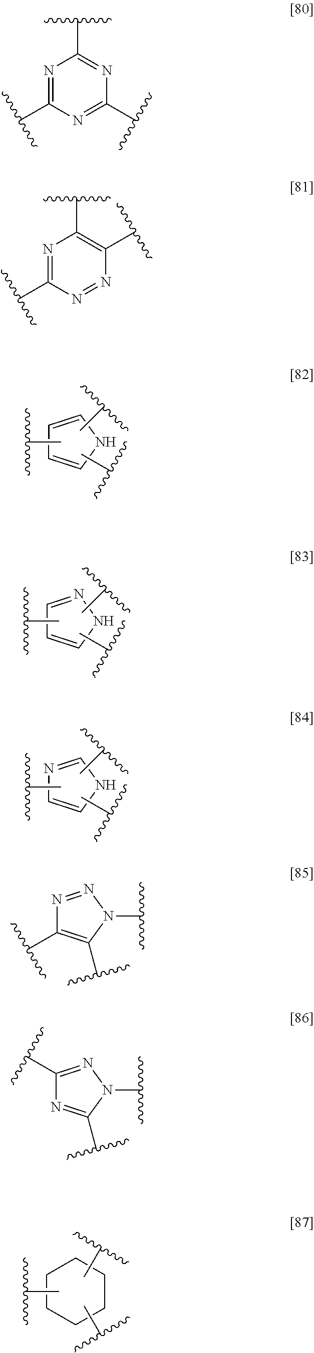

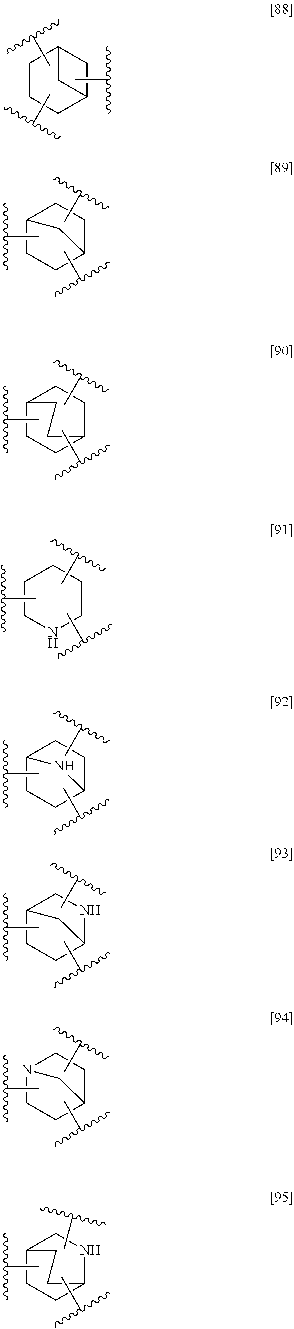

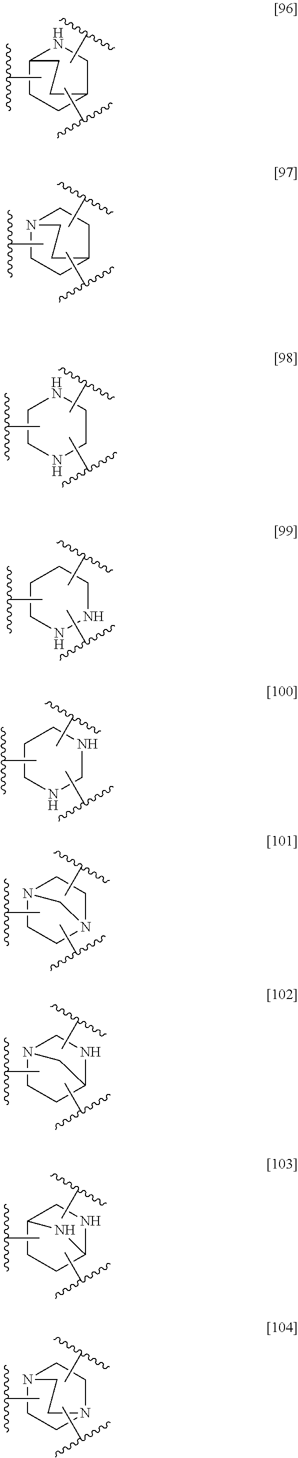

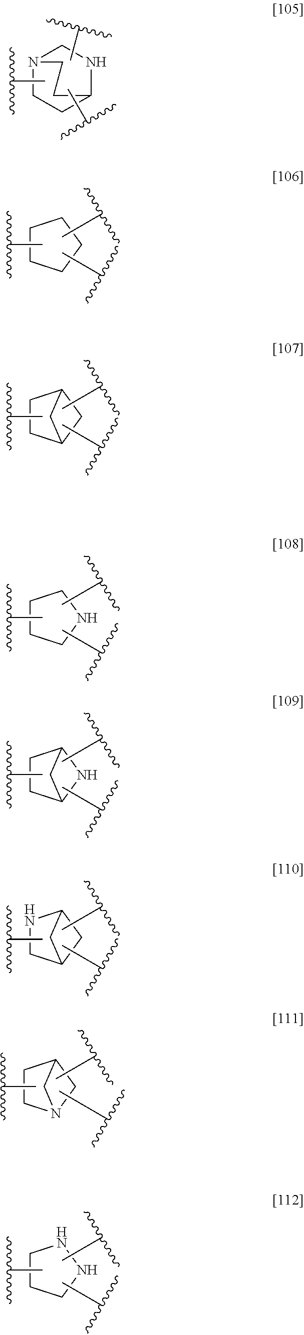

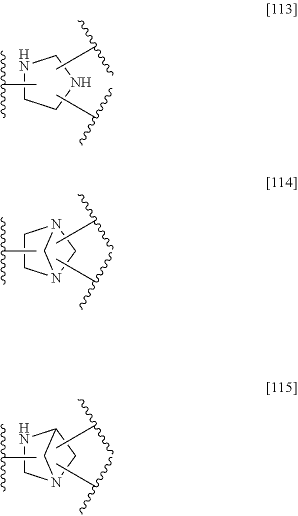

CT is a cytotoxin chosen from
   antimetabolites such as cytarabine, fludarabine, fluorouracil (5-FU), gemcitabine, methotrexate;
      alkylating cytotoxics such as adozelesin, bizelesin, carzelesin, dacarbazine (DTIC), melphalan (BCNU), temozolomide;
   mitosis inhibitors such as monomethyl auristatin E (MMAE),
   antibiotics such as dactinomycin, daunorubicin, doxorubicin, duocarmycin A, duocarmycin B1, duocarmycin B2, duocarmycin C1, duocarmycin C2, duocarmycin D, duocarmycin SA, idarubicin anthramycin, mitoxantrone;
   enzyme inhibitors such as L-asparaginase, motesanib;
   PARP inhibitors such as rucaparib, olaparib, niraparib, veliparib, iniparib;
   goserelin, leuprolide, metformin, NSC668394, tetrazole, α-solamargine, α-tomatine;
   tubulin inhibitors such as tubulysin B hydrazide;
   tyrosine kinase inhibitors such as imatinib;
   angiogenesis inhibitors such as Neovastat (AE-941);
   hedgehog signaling pathway inhibitors such as sonidegib;
   VEGFR inhibitors such as sunitinib;
   SERCA ATPase inhibitors such as thapsigargin and its derivatives 12ADT, A12ADT and S12ADT;
MG is a chelator for the complexation of a radioisotope from the group comprising ⁴³Sc, ⁴⁴Sc, ⁴⁷Sc, ⁵⁵Co, ⁶²Cu, ⁶⁴Cu, ⁶⁷Cu, ⁶⁶Ga, ⁶⁷Ga, ⁶⁸Ga, ⁸⁹Zr, ⁸⁶Y, ⁹⁰Y, ⁸⁹Zr, ⁹⁰Nb, ^{99m}Tc, ¹¹¹In, ¹³⁵Sm, ¹⁴⁰Pr, ¹⁵⁹Gd, ¹⁴⁹Tb, ¹⁶⁰Tb, ¹⁶¹Tb, ¹⁶⁵Er, ¹⁶⁶Dy, ¹⁶⁶Ho, ¹⁷⁵Yb, ¹⁷⁷Lu, ¹⁸⁶Re, ¹⁸⁸Re, ²¹¹At, ²⁰³Pb, ²¹²Pb, ²¹³Bi, ²²⁵Ac and ²³²Th;
MG is a chelator chosen from the group comprising H₄pypa, EDTA (ethylenediamine-tetraacetate), EDTMP (diethylenetriaminepenta(methylenephosphonic acid)), DTPA (diethylenetriaminepentaacetate) and derivatives thereof, NOTA (nona-1,4,7-triamine-triacetate) and derivatives thereof, such as NODAGA (1,4,7-triazacyclononane,1-glutaric acid,4,7-acetate), TRAP (triazacyclononanephosphinic acid), NOPO (1,4,7-triazacyclononane-1,4-bis[methylene(hydroxymethyl)phosphinic acid]-7-[methylene(2-carboxyethyl)phosphinic acid]), DOTA (dodeca-1,4,7,10-tetraaminetetraacetate), DOTAGA (2-(1,4,7,10-tetraazacyclododecane-4,7,10)pentanedioic acid) and other DOTA derivatives, TRITA (trideca-1,4,7,10-tetraaminetetraacetate), TETA (tetradeca-1,4,8,11-tetraaminetetraacetate) and derivatives thereof, PEPA (pentadeca-1,4,7,10,13-pentaaminepentaacetate), HEHA (hexadeca-1,4,7,10,13,16-hexaaminehexaacetate) and derivatives thereof, HBED (N,N′-bis(2-hydroxybenzyl)ethylenediamine-N,N′-diacetate) and derivatives thereof such as HBED-CC (N,N′-bis[2-hydroxy-5-carboxyethyl)benzyl)ethylenediamine-N,N′-diacetate), DEDPA and derivatives thereof, such as H₂dedpa (1,2-[[6-(carboxyl)pyridin-2-yl]methylamine]ethane) and H₄octapa (1,2-[[6-(carboxyl)pyridin-2-yl]methylamine]ethane-N,N′-diacetate), DFO (deferoxamine) and derivatives thereof, trishydroxypyridinone (THP) and derivatives thereof such as H₃THP-Ac and H₃THP-mal (YM103), TEAP (tetraazycyclodecanephosphinic acid) and derivatives thereof, AAZTA (6-amino-6-methylperhydro-1,4-diazepane-N,N,N′,N′-tetraacetate) and derivatives thereof, such as AAZTA⁵ (5-[(6-amino)-1,4-diazepane]pentanoic acid-N,N,N′,N′-tetraacetate), DATA (2,2′-(6-((carboxymethyl)(methyl)amino)-6-methyl-1,4-diazepane-1,4-diyl)diacetate) and derivatives thereof, such as DATA⁵m (5-[[6-(N-methyl)amino]-1,4-diacetate-1,4-diazepane]pentanoic acid-N,N′,N′-triacetate); sarcophagine SAR (1-N-(4-aminobenzyl)-3,6,10,13,16,19-hexaazabicyclo[6.6.6]-eicosane-1,8-diamine) and derivatives thereof, such as (NH₂)₂SAR (1,8-diamino-3,6,10,13,16,19-hexaazabicyclo[6.6.6]eicosane), N4 (3-[(2′-aminoethyl)amino]-2-[(2″-aminoethyl)-aminomethyl]propionic acid) and other N₄ derivatives, PnAO (6-(4-isothiocyanatobenzyl)-3,3,9,9-tetramethyl-4,8-diazaundecane-2,10-dione dioxime) and derivatives such as BMS181321 (3,3′-(1,4-butanediyldiamino)-bis(3-methyl-2-butanone) dioxime), MAG2 (mercaptoacetylglycylglycine) and derivatives thereof, MAG3 (mercaptoacetylglycylglycylglycine) and derivatives thereof, such as N₃S adipate, MAS3 (mercaptoacetylserylserylserine) and derivatives thereof, MAMA (N-(2-mercaptoethyl)-2-[(2-mercaptoethyl)amino]acetamide) and derivatives thereof, EC (ethylenedicysteine) and derivatives thereof, dmsa (dimercaptosuccinic acid) and derivatives thereof, DADT (diaminodithiol), DADS (diaminodisulfide), N₂S₂ chelators and derivatives thereof, aminothiols and derivatives thereof; salts of the aforementioned chelators; hydrazinenicotinamide (HYNIC) and hydrazinenicotinamide derivatives;
MG is DOTA (dodeca-1,4,7,10-tetraaminetetraacetate);
MG is DOTAGA (2-(1,4,7,10-tetraazacyclododecane-4,7,10)pentanedioic acid);
MG is DATA^{5m} (1,4-bis(carboxymethyl)-6-[methylcarboxymethylamino]-6-pentanoic acid-1,4-diazepane);
MG is AAZTA⁵ (1,4-bis(carboxymethyl)-6-[bis(carboxymethyl)amino]-6-pentanoic acid-1,4-diazepane);
MG is a labeling group for the covalent binding of ¹⁸F, ¹³¹I or ²¹¹At;
MG is chosen from

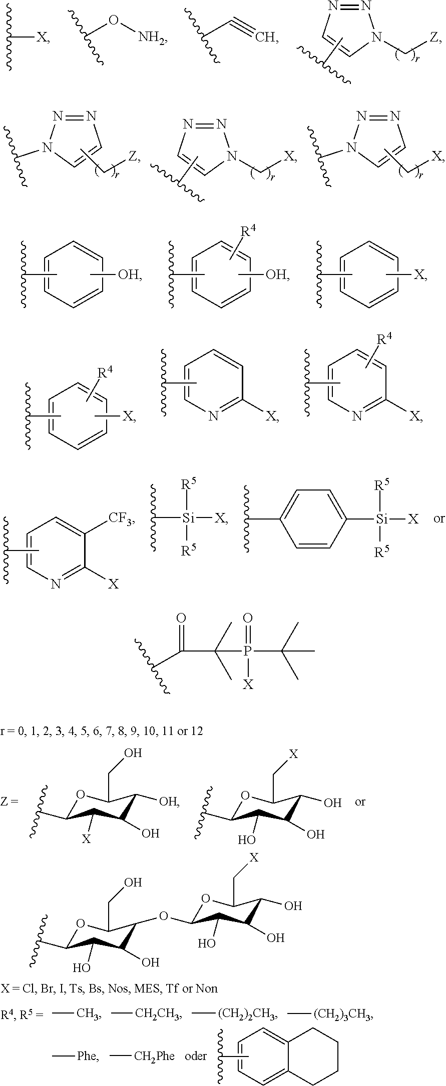

MG is a group of the |-CF₂—X type with a leaving group X for substitution by ¹⁸F, ¹³¹I or ²¹¹At; and/or
MG contains a leaving group X chosen from a radical of bromine (Br), chlorine (Cl) or iodine (1), tosyl (Ts), brosylate (Bs), nosylate (Nos), 2-(N-morpholino)ethanesulfonic acid (MES), triflate (Tf) and nonaflate (Non).

The invention additionally relates to radiotracers comprising one of the above-described pharmaceutical compounds and a radioisotope complexed or conjugated therewith. In an advantageous embodiment of the invention, the radiotracer comprises one of the above-described pharmaceutical compounds and a radioisotope complexed therewith, chosen from the group comprising ⁴⁴Sc, ⁴⁷Sc, ⁵⁵Co, ⁶²Cu, ⁶⁴Cu, ⁶⁷Cu, ⁶⁶Ga, ⁶⁷Ga, ⁶⁸Ga, ⁸⁹Zr, ⁸⁶Y, ⁹⁰Y, ⁸⁹Zr, ⁹⁰Nb, ^{99m}Tc, ¹¹¹In ¹³⁵Sm, ¹⁴⁰Pr ¹⁵⁹Gd, ¹⁴⁹Tb, ¹⁶⁰Tb, ¹⁶¹Tb, ¹⁶⁵Er, ¹⁶⁶Dy, ¹⁶⁶Ho, ¹⁷⁵Yb, ¹⁷⁷Lu, ¹⁸⁶Re, ¹⁸⁸Re, ²¹¹At, ²⁰³Pb, ²¹²Pb, ²¹³Bi, ²²⁵Ac and ²³²Th.

The radiotracer preferably comprises one of the above-described pharmaceutical compounds and the radioisotope ⁶⁸Ga complexed therewith.

In particular, the radiotracer comprises one of the above-described pharmaceutical compounds and the radioisotope ¹⁷⁷Lu complexed therewith.

In a further advantageous embodiment of the invention, the radiotracer comprises one of the above-described pharmaceutical compounds and a radioisotope conjugated therewith, chosen from the group comprising ¹⁸F, ¹³¹I and ²¹¹At.

Pharmaceutical compounds of the invention include an FAP inhibitor, an FAP substrate and a cytotoxin, and optionally a labeling group for a radioisotope. The high affinity of known FAP inhibitors (antagonists) is combined here with the action of FAP substrates in order to deposit the cytotoxin selectively and in high dose in the tumor stroma and optionally via endocytosis in cancer-associated fibroblasts (CAFs) and carcinogenic cells. Because of its prevalent expression in many cancers, the invention uses the fibroblast activation protein (FAP) simultaneously
as biological target;
for enzymatic cleavage of the FAPs-cytotoxin conjugate;
for release of the cytotoxin in its pharmacologically active form.

Subsequently, the cytotoxin can be internalized and become cytotoxically active.

In an advantageous embodiment, the compound of the invention comprises a labeling group for a diagnostic or therapeutic radioisotope, for example ⁶⁸Ga, ⁹⁰Y, ¹⁷⁷Lu or ²²⁵Ac. This enables
more or less direct translation from diagnosis to therapy;
combined cytotoxic and radiological insult of CAFs and carcinogenic cells; and
quantitative evaluation of the success of treatment by means of downstream imaging diagnosis.

Because of the simultaneous insult of CAFs and carcinogenic cells, efficacy is increased, the patient population is extended, and clinical use and efficiency are considerably facilitated and enhanced.

The FAP inhibitors (FAPi) used in accordance with the invention have high binding affinity for the fibroblast activation protein (FAP), which is overexpressed in various cancers on cancer-associated fibroblasts (CAFs). In addition, an FAP substrate (FAPs) is used, which is conjugated to a cytotoxin (CT) via an FAP-cleavable amide bond. This enables use of the pharmaceuticals of the invention by the concept of targeted drug delivery. The pharmaceutical of the invention functions here as a prodrug, from which the cytotoxin (CT) is released and “activated” on contact with FAP. Because of the combination of FAPi and FAPs, the pharmaceuticals of the invention have a crucially elevated affinity for FAP by comparison with known targeted drug delivery systems with simple FAP substrates (FAPs). Accordingly, the pharmaceuticals of the invention have higher and more selective accumulation in the tumor tissue or stroma; this is associated with reduced accumulation in healthy tissue.

The linkers L1, L2, L3, L4, L5, L6 function simultaneously as spacer and chemical modulator that compensates for impairment of the biochemical function of the targeting vectors (binding affinity for the target) and of the radiochemical function of the labeling group (e.g. stable complexation of the radionuclide by the chelator), for example owing to steric hindrance.

The tris linker TL ensures that a labeling group MG—especially a chelator—is covalently bonded to the rest of the molecules solely via a functional group, as in known monomeric radiopharmaceuticals. Accordingly, by comparison with established radiotracers, labeling properties are maintained essentially unchanged. This ensures high labeling effectiveness and in vivo stability of the radiopharmaceuticals of the invention.

In an advantageous embodiment, the radiopharmaceuticals of the invention are intended for labeling with the radioisotopes ¹⁸F, ¹²³I, ¹²⁴I, ¹²⁵I, ¹³¹I and ²¹¹At. Accordingly, the labeling group MG contains a leaving group X which is substituted by one of the radioisotopes ¹⁸F, ¹²³I, ¹²⁴I, ¹²⁵I, ¹³¹I and ²¹¹At.

In a further advantageous embodiment, the radiopharmaceuticals of the invention are intended for labeling one of the metallic radioisotopes ⁴⁴Sc, ⁴⁷Sc, ⁵⁵Co, ⁶²Cu, ⁶⁴Cu, ⁶⁷Cu, ⁶⁶Ga, ⁶⁷Ga, ⁶⁸Ga, ⁸⁹Zr, ⁸⁶Y, ⁹⁰Y ⁸⁹Zr, ⁹⁰Nb, ^{99m}Tc, ¹¹¹In ¹³⁵Sm ¹⁴⁰Pr ¹⁵⁹Gd, ¹⁴⁹Tb, ¹⁶⁰Tb, ¹⁶¹Tb, ¹⁶⁵Er, ¹⁶⁶Dy ¹⁶⁶Ho, ¹⁷⁵Yb, ¹⁷⁷Lu, ¹⁸⁶Re, ¹⁸⁸Re, ²¹¹At, ²⁰³Pb, ²¹²Pb, ²¹³Bi, ²²⁵Ac and ²³²Th. Accordingly, the labeling group MG contains a chelator for complexation of one of the radioisotopes ⁴⁴Sc, ⁴⁷Sc, ⁵⁵Co, ⁶²Cu, ⁶⁴Cu, ⁶⁷Cu, ⁶⁶Ga, ⁶⁷Ga, ⁶⁸Ga, ⁸⁹Zr, ⁸⁶Y, ⁹⁰Y, ⁸⁹Zr, ⁹⁰Nb, ^{99m}Tc, ¹¹¹1n, ¹³⁵Sm, ¹⁴⁰Pr ¹⁵⁹Gd, ¹⁴⁹Tb, ¹⁶⁰Tb, ¹⁶¹Tb, ¹⁶⁵Er, ¹⁶⁶Dy ¹⁶⁶Ho, ¹⁷⁵Yb, ¹⁷⁷Lu, ¹⁸⁶Re, ¹⁸⁸Re, ²¹¹At, ²⁰³Pb, ²¹²Pb, ²¹³Bi, ²²⁵Ac and ²³²Th.

### Chelators

The prior art discloses a multitude of chelators for the complexation of the above radioisotopes. Scheme 5 shows examples of chelators used in accordance with the invention.

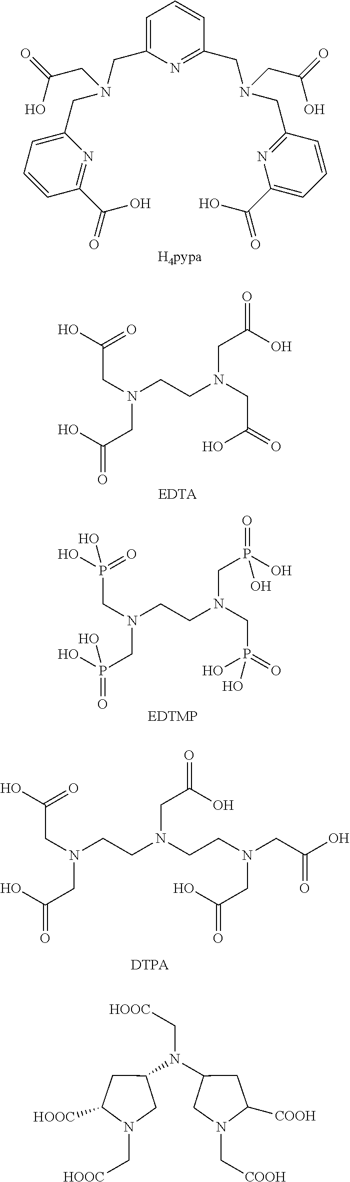 [SMILES: O=C(O)CN(Cc1cccc(CN(CC(=O)O)Cc2cccc(C(=O)O)n2)n1)Cc1cccc(C(=O)O)n1 ; O=C(O)CN(CCN(CC(=O)O)CC(=O)O)CC(=O)O ; O=P(O)(O)CN(CCN(CP(=O)(O)O)CP(=O)(O)O)CP(=O)(O)O ; O=C(O)CN(CCN(CC(=O)O)CC(=O)O)CCN(CC(=O)O)CC(=O)O ; O=C(O)CN1CC(N(CC(=O)O)[C@H]2C[C@@H](C(=O)O)N(CC(=O)O)C2)CC1C(=O)O]

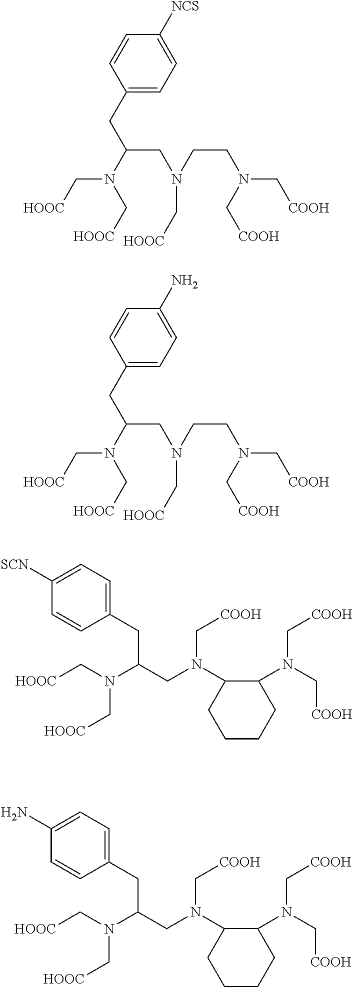

### Stabilized DTPA Derivatives

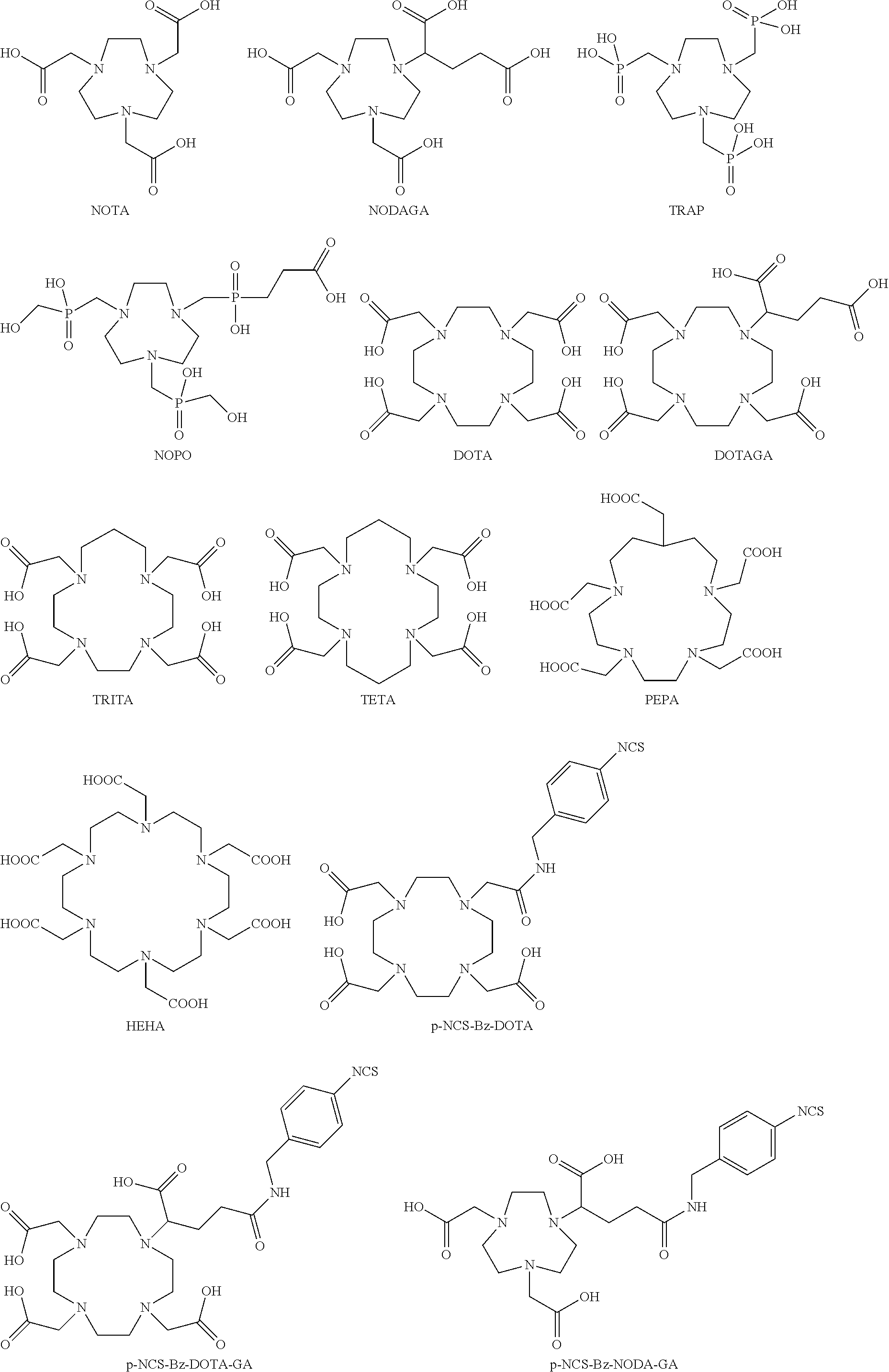

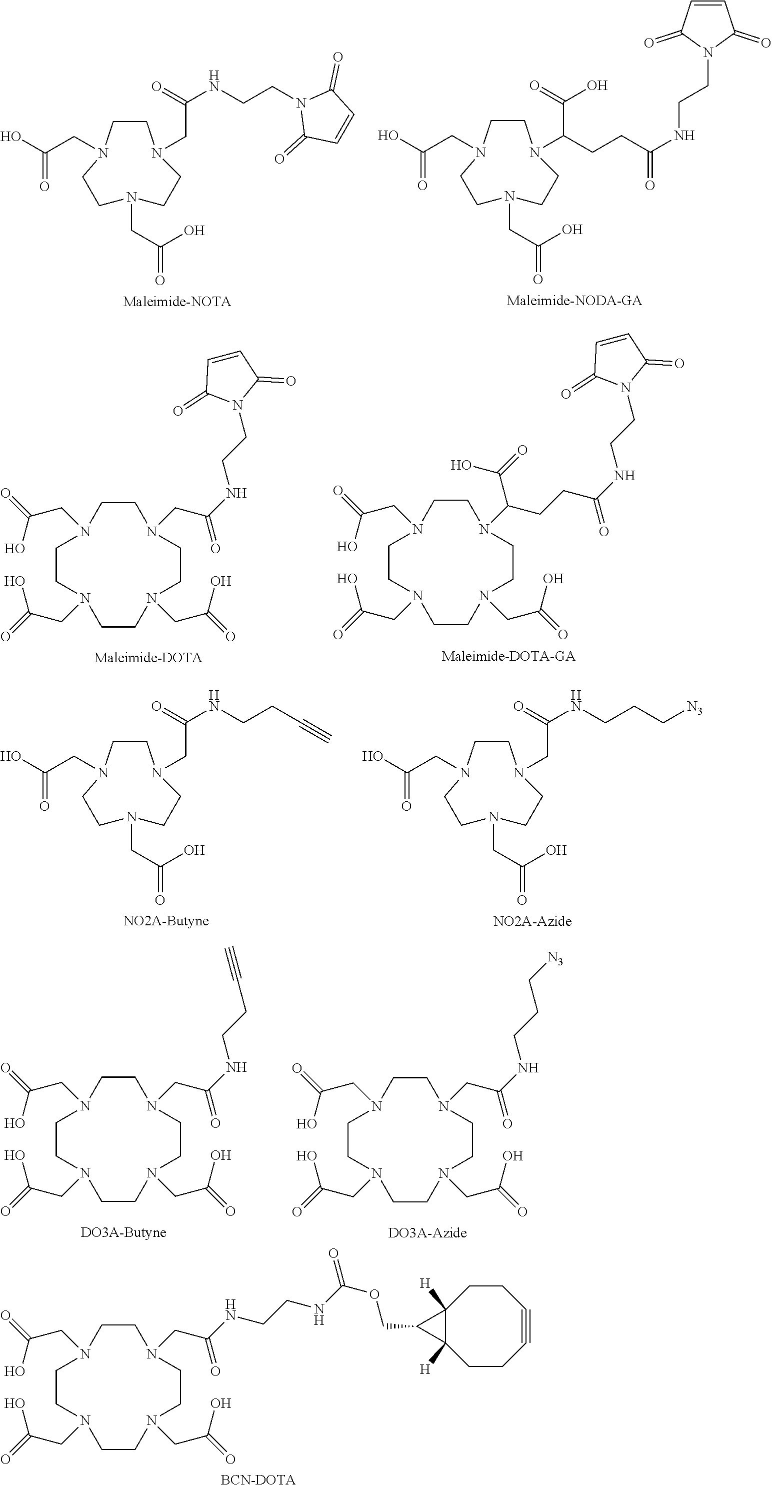

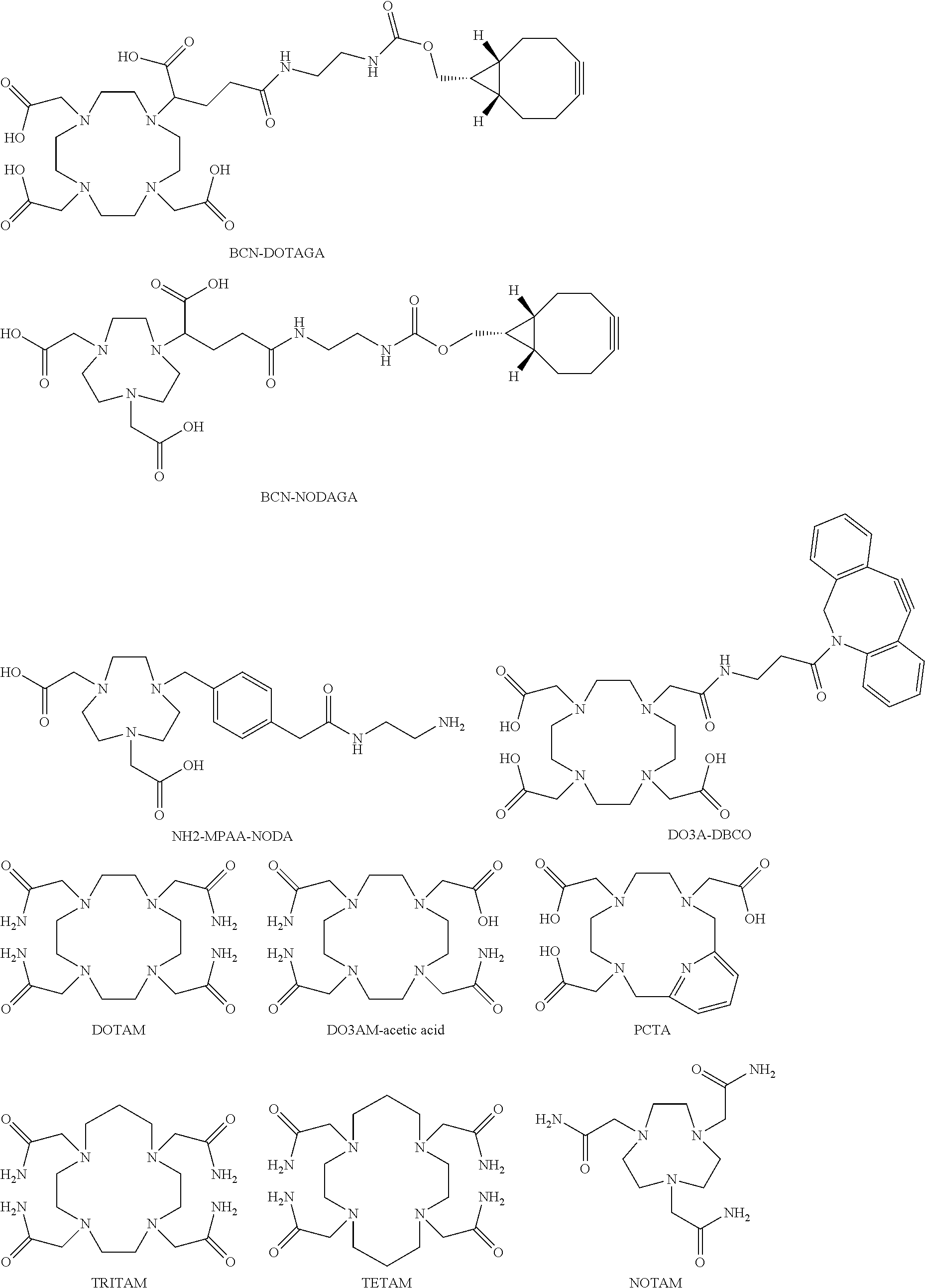

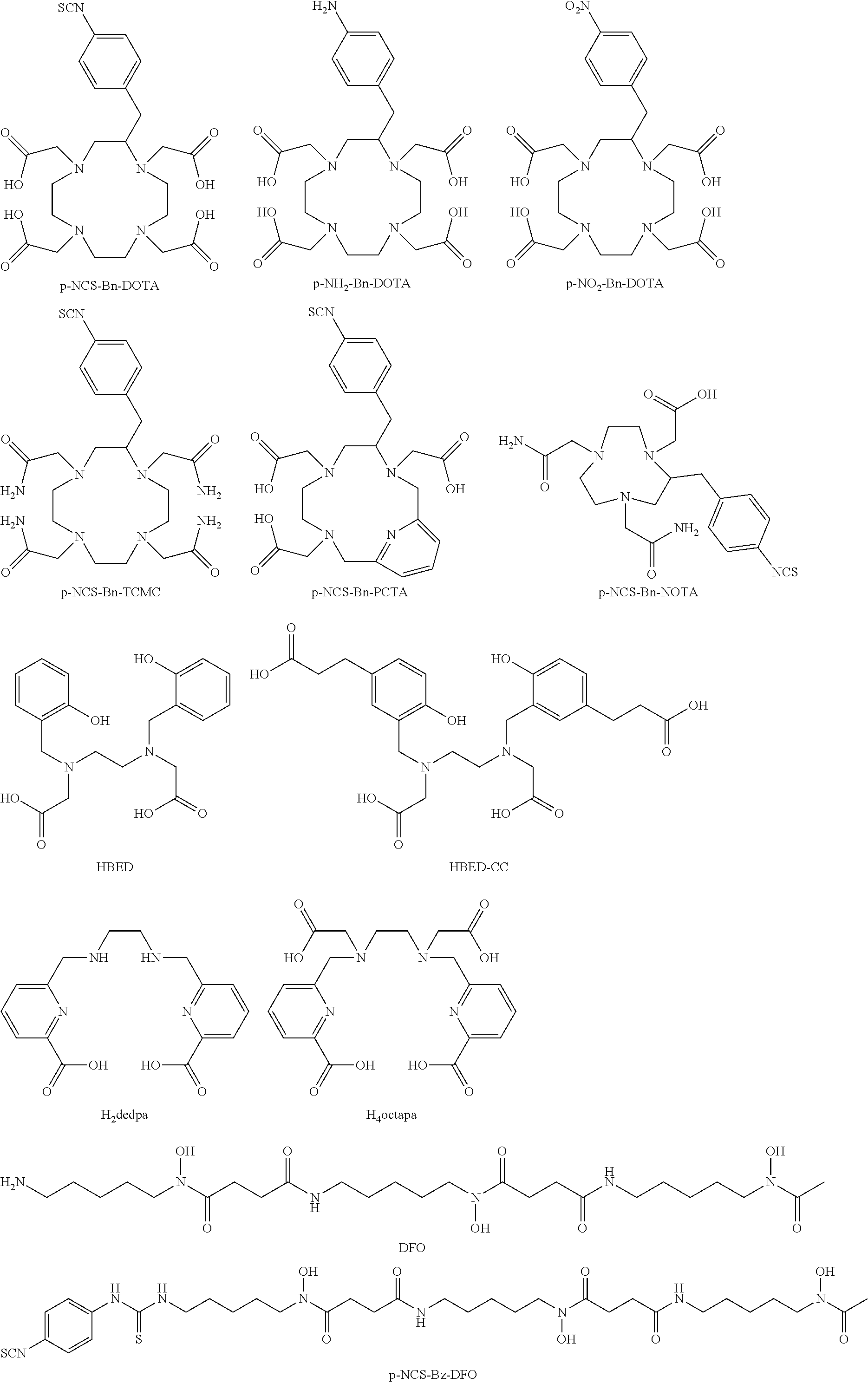

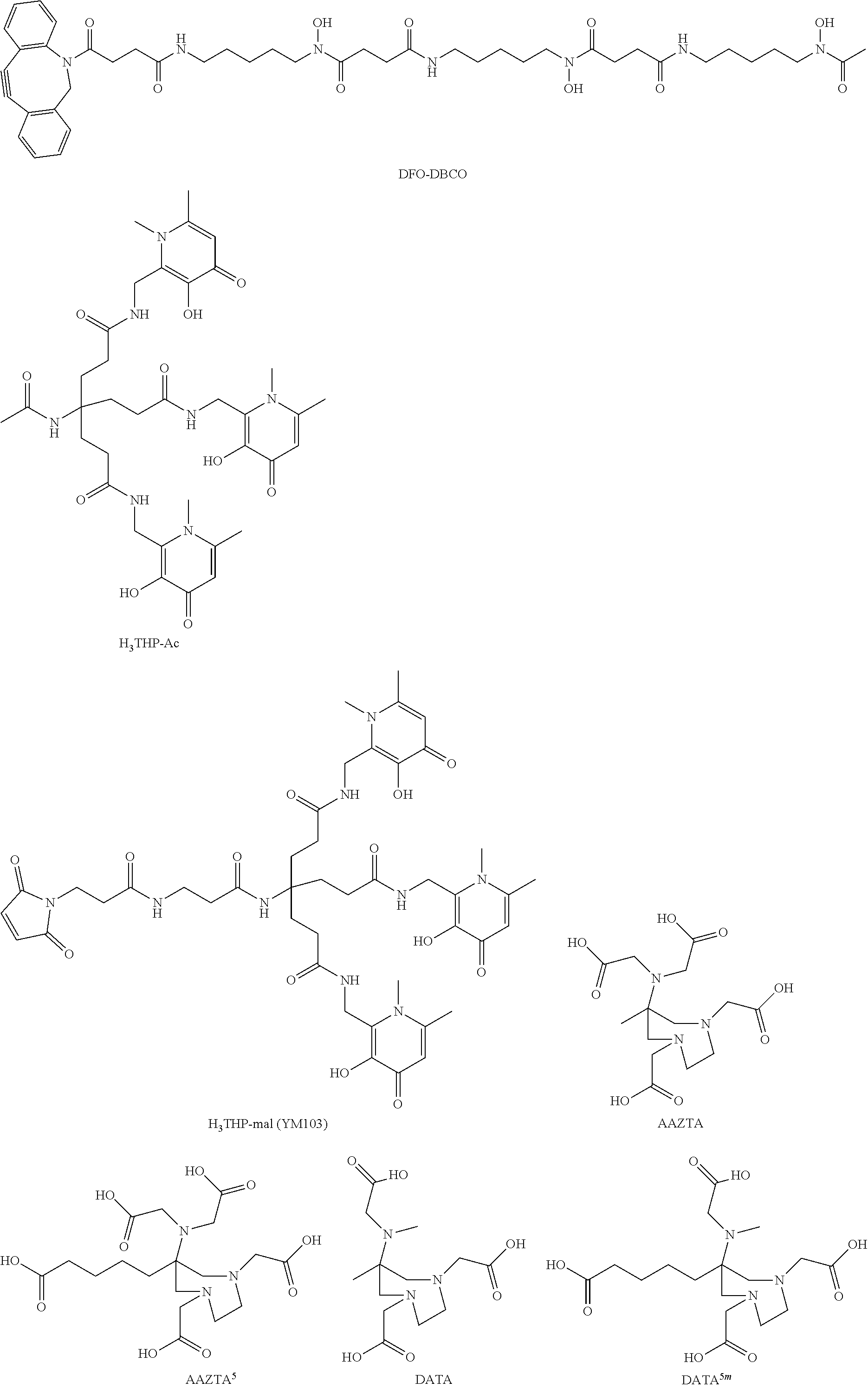

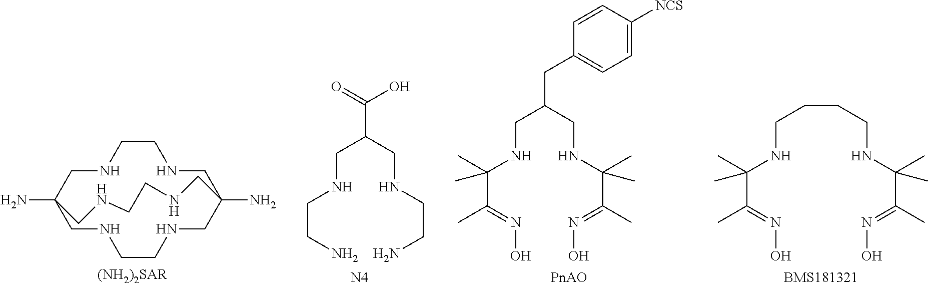

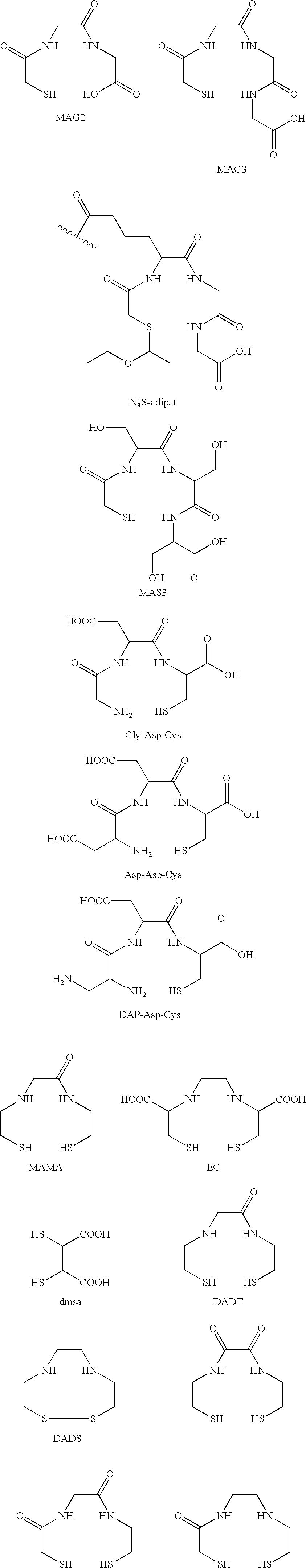

### HYNIC Derivatives

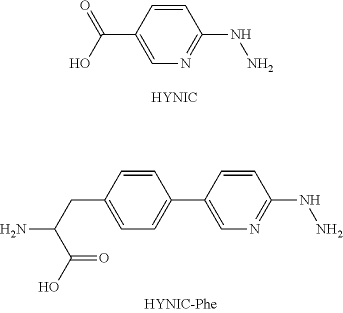

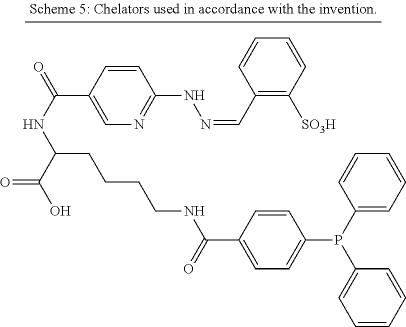 [SMILES: O=C(NCCCCC(NC(=O)c1ccc(N/N=C\c2ccccc2S(=O)(=O)O)nc1)C(=O)O)c1ccc(P(c2ccccc2)c2ccccc2)cc1]

### Cytotoxin (Cytostatic) CT

The prior art discloses a multitude of cytotoxic active ingredients for cancer treatment. For example, rucaparib and some of its derivatives inhibit the enzyme PARP (poly-ADP-ribose polymerase), which is involved in the repair of single-strand breaks (SSBs) in DNA. The effect of PARP inhibitors is based on synthetically induced lethality. In a healthy cell with intact DNA repair, PARP inhibition does not lead to cell death because double-strand breaks (DSB) in the DNA that have resulted from SSBs are repaired by homologous recombination (HR). In HR-deficient cells, by contrast, PARP inhibition leads to cell death since DSBs accumulate in the cell and recruit apoptosis molecules. The two genes BRCA1 and BRCA2 (breast cancer gene) are crucially involved in HR. A mutation in these genes leads to malfunction of DNA repair and increases the risk of tumor formation.

In 20-25% of patients with mCRPC (metastasized castration-resistant prostate carcinoma), HR genes, including BRCA1/2, have mutated. These patients benefit from treatment with PARP inhibitors, which have high tumor specificity. It is also possible to pharmaceutically induce BRCA deficiency. The active ingredient enzalutamide, an inhibitor of the androgen receptor signaling pathway, is able to bring about downregulation of the BRCA genes. After administration of enzalutamide, even patients without BRCA mutation can benefit from the selective tumor toxicity of rucaparib. The patient population for PARP therapy can thus be extended.

Temozolomide is a pharmaceutically adapted active ingredient (prodrug) which, after metabolization and spontaneous hydrolytic cleavage, releases methylhydrazine (CH₃(NH)NH₂), which methylates DNA bases and induces apoptosis.

Monomethyl auristatin E (MMAE) is an antineoplastic active ingredient that interrupts the cell cycle by inhibition of tubulin polymerization and hence leads to apoptosis.

Table 1 shows cytotoxins (cytostatics) used in accordance with the invention. Preference is given to using cytotoxins that have a primary or secondary amino group and form an FAP-cleavable amide bond.

TABLE 1
Cytotoxins (CT) used in accordance with the invention

| | |
|---|---|
| Adozelesin | 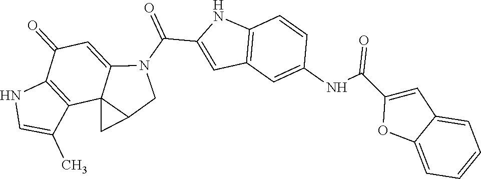 [SMILES: Cc1c[nH]c2c1C13CC1CN(C(=O)c1cc4cc(NC(=O)c5cc6ccccc6o5)ccc4[nH]1)C3=CC2=O] |
| Anthramycin | 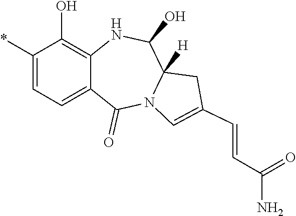 [SMILES: *c1ccc2c(c1O)N[C@@H](O)[C@@H]1CC(/C=C/C(N)=O)=CN1C2=O] |
| Bizelesin | 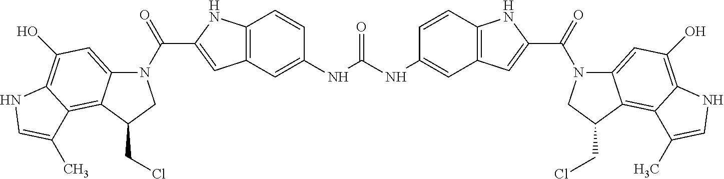 [SMILES: Cc1c[nH]c2c(O)cc3c(c12)[C@H](CCl)CN3C(=O)c1cc2cc(NC(=O)Nc3ccc4[nH]c(C(=O)N5C[C@@H](CCl)c6c5cc(O)c5[nH]cc(C)c65)cc4c3)ccc2[nH]1] |
| Carzelesin | 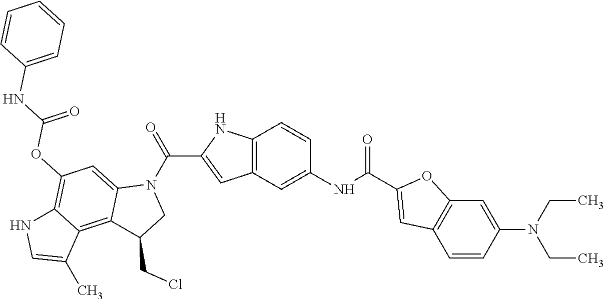 [SMILES: CCN(CC)c1ccc2cc(C(=O)Nc3ccc4[nH]c(C(=O)N5C[C@@H](CCl)c6c5cc(OC(=O)Nc5ccccc5)c5[nH]cc(C)c65)cc4c3)oc2c1] |
| CC-1065 | 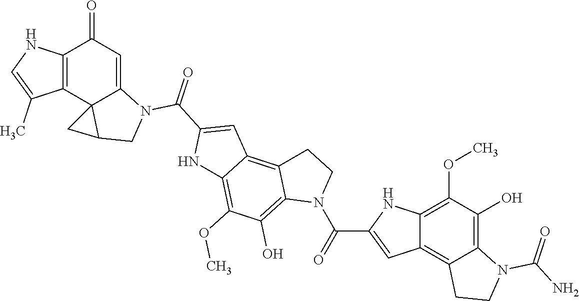 [SMILES: COc1c(O)c2c(c3cc(C(=O)N4CCc5c4c(O)c(OC)c4[nH]c(C(=O)N6CC7CC78C6=CC(=O)c6[nH]cc(C)c68)cc54)[nH]c13)CCN2C(N)=O] |
| Cytarabine (ara-C) | 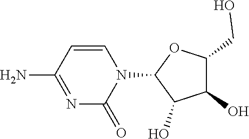 [SMILES: Nc1ccn([C@@H]2O[C@H](CO)[C@@H](O)[C@@H]2O)c(=O)n1] |
| Dacarbazine (DTIC) | 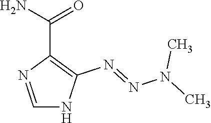 |
| Dactinomycin | 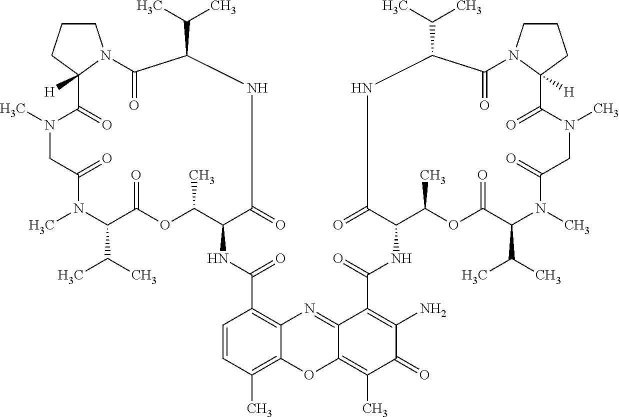 |
| Daunorubicin | 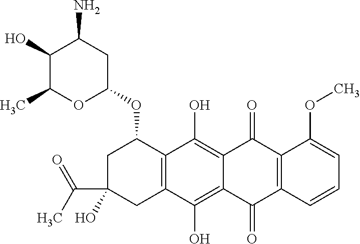 [SMILES: COc1cccc2c1C(=O)c1c(O)c3c(c(O)c1C2=O)C[C@@](O)(C(C)=O)C[C@@H]3O[C@H]1C[C@H](N)[C@H](O)[C@H](C)O1] |
| Doxorubicin | 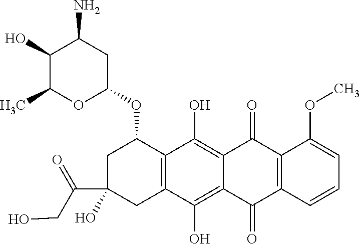 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1] |
| Duocarmycin A | 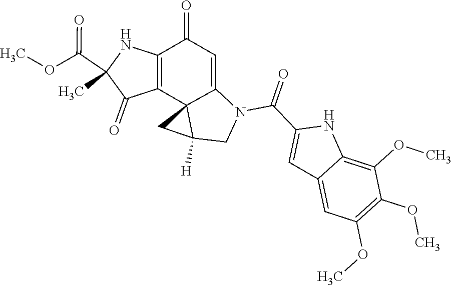 |
| Duocarmycin B1 | 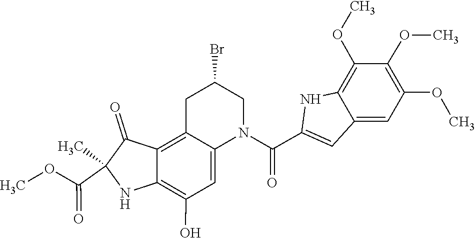 |
| Duocarmycin B2 | 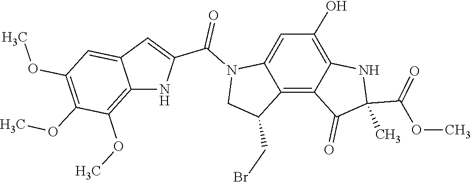 |
| Duocarmycin C1 | 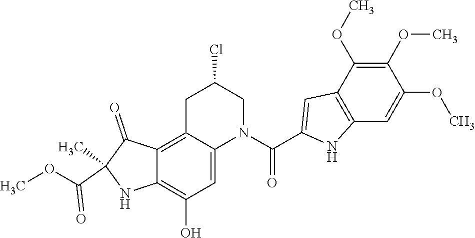 |
| Duocarmycin C2 | 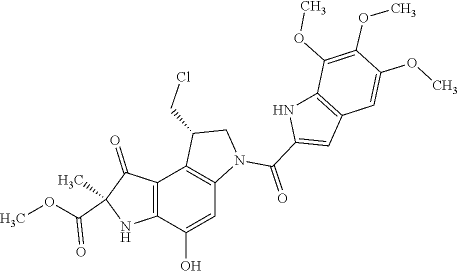 |
| Duocarmycin D | 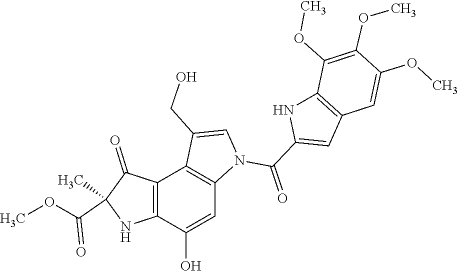 |
| Duocarmycin SA | 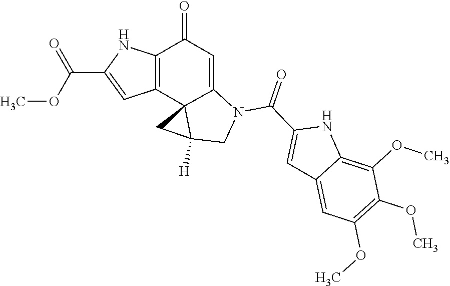 |
| Fludarabine | 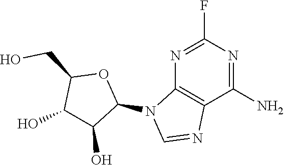 [SMILES: Nc1nc(F)nc2c1ncn2[C@@H]1O[C@H](CO)[C@@H](O)[C@@H]1O] |
| Fluorouracil (5-FU) | 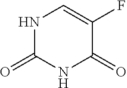 |
| Gemcitabine | 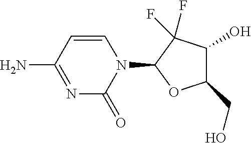 [SMILES: Nc1ccn([C@@H]2O[C@H](CO)[C@@H](O)C2(F)F)c(=O)n1] |
| Goserelin | 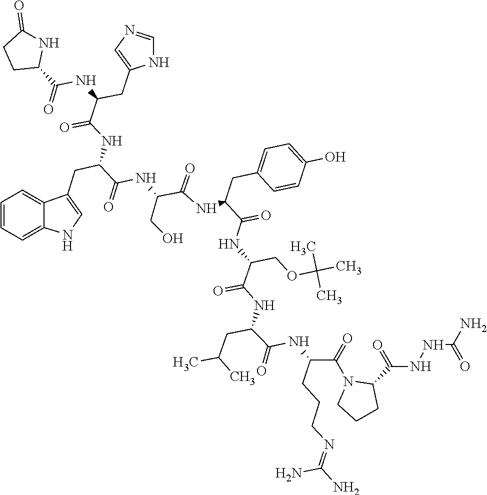 [SMILES: CC(C)C[C@H](NC(=O)[C@@H](COC(C)(C)C)NC(=O)[C@H](Cc1ccc(O)cc1)NC(=O)[C@H](CO)NC(=O)[C@H](Cc1c[nH]c2ccccc12)NC(=O)[C@H](Cc1cnc[nH]1)NC(=O)[C@@H]1CCC(=O)N1)C(=O)N[C@@H](CCCN=C(N)N)C(=O)N1CCC[C@H]1C(=O)NNC(N)=O] |
| Idarubicin | 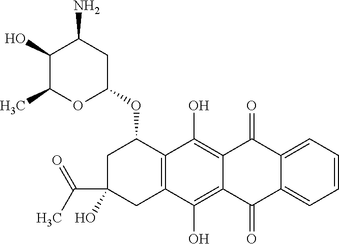 [SMILES: CC(=O)[C@]1(O)Cc2c(O)c3c(c(O)c2[C@@H](O[C@H]2C[C@H](N)[C@H](O)[C@H](C)O2)C1)C(=O)c1ccccc1C3=O] |
| Imatinib | 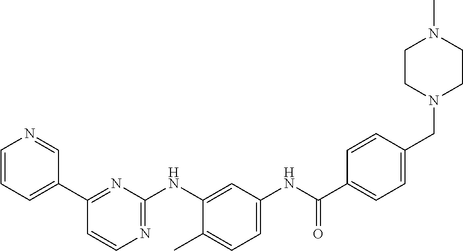 [SMILES: Cc1ccc(NC(=O)c2ccc(CN3CCN(C)CC3)cc2)cc1Nc1nccc(-c2cccnc2)n1] |
| L-Asparaginase* | MEFFKKTALAALVMGFSGAALALPNITILATGGTIAGGGDSATKSNYTVGKVGVENLVNA |
| | VPQLKDIANVKGEQVVNIGSQDMNDNVWLTLAKKINTDCDKTDGFVITHGTDTMEETAYF |
| | LDLTVKCDKPVVMVGAMRPSTSMSADGPFNLYNAVVTAADKASANRGVLVVMNDTVLDGR |
| | DVTKTNTTDVATFKSVNYGPLGYIHNGKIDYQRTPARKHTSDTPFDVSKLNELPKVGIVY |
| | NYANASDLPAKALVDAGYDGIVSAGVGNGNLYKSVEDTLATAAKTGTAVVRSSRVPTGAT |
| | TQDAEVDDAKYGFVASGTLNPQKARVLLQLALTQTKDPOQIQQI FNQY |
| Leuprolide* | PHWSYLLR |
| Melphalan (BCNU) | 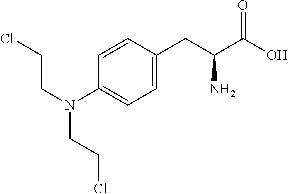 [SMILES: N[C@@H](Cc1ccc(N(CCCl)CCCl)cc1)C(=O)O] |
| Metformin | 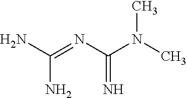 |
| Methotrexate | 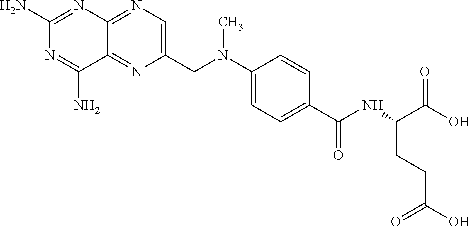 [SMILES: CN(Cc1cnc2nc(N)nc(N)c2n1)c1ccc(C(=O)N[C@@H](CCC(=O)O)C(=O)O)cc1] |
| Mitoxantrone | 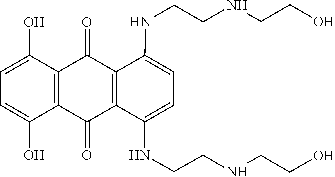 [SMILES: O=C1c2c(O)ccc(O)c2C(=O)c2c(NCCNCCO)ccc(NCCNCCO)c21] |
| Monomethyl auristatin E (MMAE) | 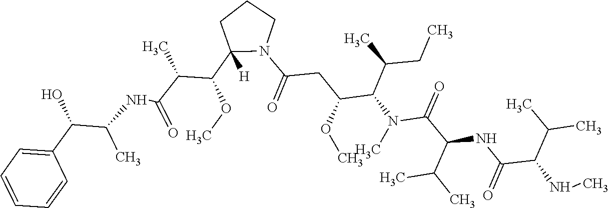 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C] |
| Motesanib | 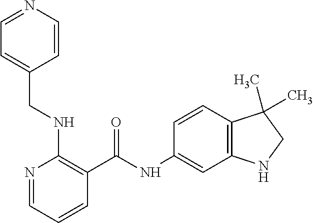 [SMILES: CC1(C)CNc2cc(NC(=O)c3cccnc3NCc3ccncc3)ccc21] |
| Neovastat (AE-941) | 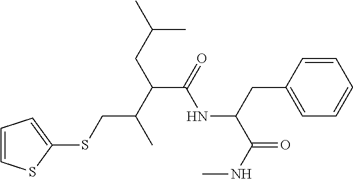 [SMILES: CNC(=O)C(Cc1ccccc1)NC(=O)C(CC(C)C)C(C)CSc1cccs1] |
| NSC668394 | 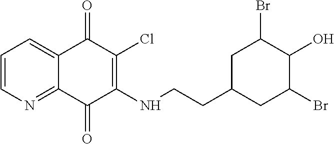 [SMILES: O=C1C(Cl)=C(NCCC2CC(Br)C(O)C(Br)C2)C(=O)c2ncccc21] |
| Rucaparib | 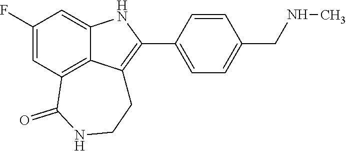 [SMILES: CNCc1ccc(-c2[nH]c3cc(F)cc4c3c2CCNC4=O)cc1] |
| Temozolomide | 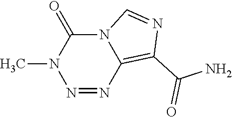 |
| Tetrazole | 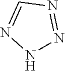 |
| Tubulysin B hydrazide | 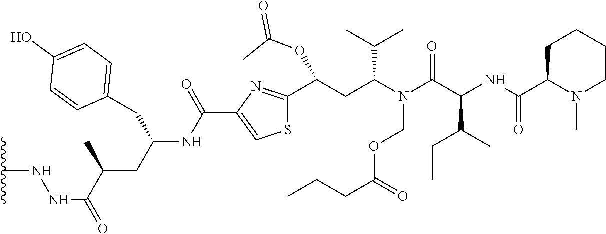 [SMILES: *NNC(=O)[C@@H](C)C[C@H](Cc1ccc(O)cc1)NC(=O)c1csc([C@@H](C[C@H](C(C)C)N(COC(=O)CCC)C(=O)[C@@H](NC(=O)[C@H]2CCCCN2C)C(C)CC)OC(C)=O)n1] |
| α-Solamargine | 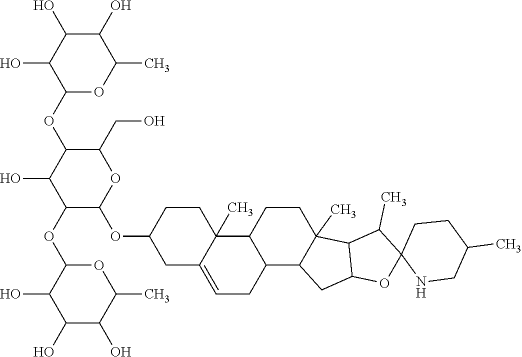 [SMILES: CC1CCC2(NC1)OC1CC3C4CC=C5CC(OC6OC(CO)C(OC7OC(C)C(O)C(O)C7O)C(O)C6OC6OC(C)C(O)C(O)C6O)CCC5(C)C4CCC3(C)C1C2C] |
| Sonidegib | 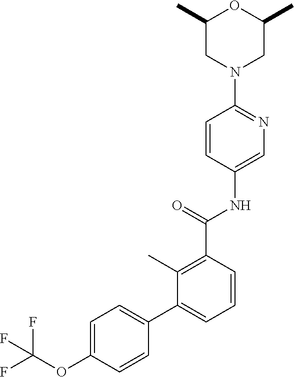 [SMILES: Cc1c(C(=O)Nc2ccc(N3C[C@@H](C)O[C@@H](C)C3)nc2)cccc1-c1ccc(OC(F)(F)F)cc1] |
| Sunitinib | 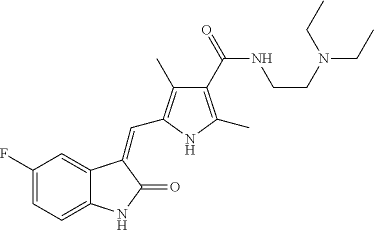 [SMILES: CCN(CC)CCNC(=O)c1c(C)[nH]c(/C=C2\C(=O)Nc3ccc(F)cc32)c1C] |
| Thapsigargin (and d erivatives thereof) | 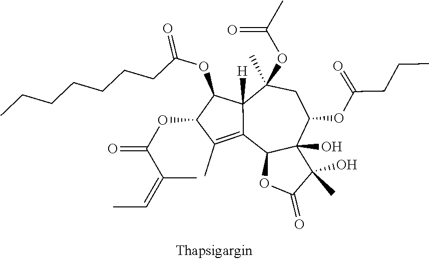 [SMILES: C/C=C(/C)C(=O)O[C@H]1C(C)=C2[C@H]([C@@H]1OC(=O)CCCCCCC)[C@@](C)(OC(C)=O)C[C@H](OC(=O)CCC)[C@@]1(O)[C@H]2OC(=O)[C@@]1(C)O] |
| | 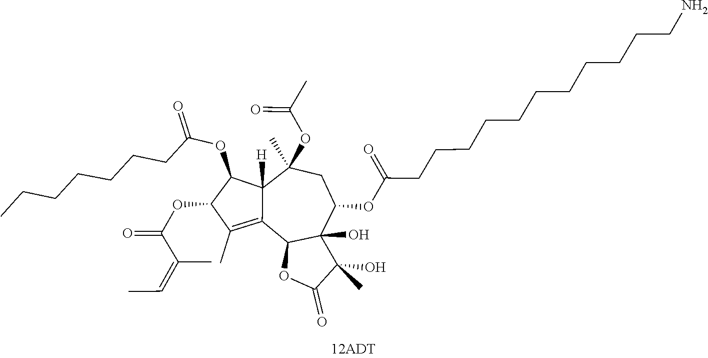 [SMILES: C/C=C(/C)C(=O)O[C@H]1C(C)=C2[C@H]([C@@H]1OC(=O)CCCCCCC)[C@@](C)(OC(C)=O)C[C@H](OC(=O)CCCCCCCCCCCN)[C@@]1(O)[C@H]2OC(=O)[C@@]1(C)O] |
| | 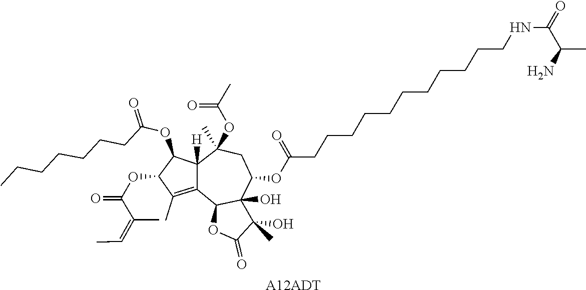 |
| | 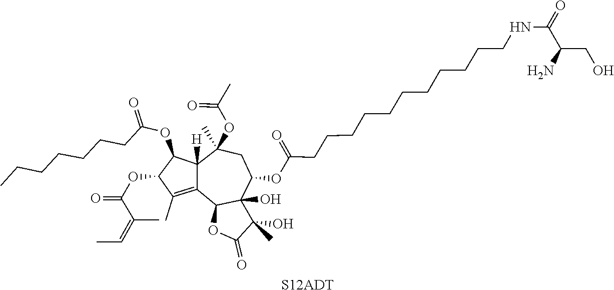 |
| α-Tomatine | 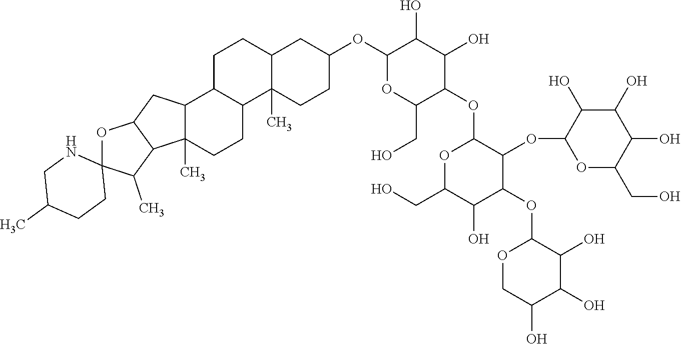 [SMILES: CC1CCC2(NC1)OC1CC3C4CCC5CC(OC6OC(CO)C(OC7OC(CO)C(O)C(OC8OCC(O)C(O)C8O)C7OC7OC(CO)C(O)C(O)C7O)C(O)C6O)CCC5(C)C4CCC3(C)C1C2C] |

*Peptide with amino acid sequence

### Amide Coupling

In the invention, functional groups, such as the labeling group MG, FAPi, FAPs, the bivalent linkers L1, L2, L3, L4, L5, L6 and the tris linker TL, are preferably conjugated by an amide coupling reaction. In advantageous embodiments of the invention, in particular, the coupling groups K1, K2, K3, K4, K5 are configured as amides.

The most commonly used reaction in medicinal chemistry is amide coupling, which forms the backbone of proteins. A general example of amide coupling is shown in scheme 6.

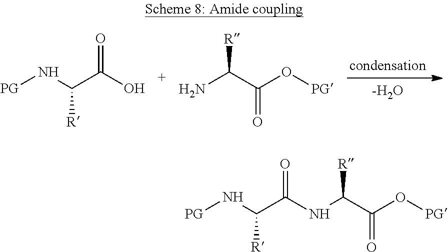

Because there is a virtually unlimited set of readily available carboxylic acid derivatives and amine derivatives, amide coupling strategies open up a simple route for the synthesis of new compounds. The person skilled in the art is aware of numerous reagents and protocols for amide couplings. The most commonly used amide coupling strategy is based on the condensation of a carboxylic acid with an amine. For this purpose, the carboxylic acid is generally activated. Before the activation, remaining functional groups are protected. The reaction is effected in two steps, either in one reaction medium (single pot) with direct conversion of the activated carboxylic acid or in two steps with isolation of an activated “trapped” carboxylic acid and reaction with an amine.

The carboxylic acid reacts here with a coupling reagent to form a reactive intermediate, which can be isolated or reacted directly with an amine. Numerous reagents are available for carboxylic acid activation, such as acid halides (chloride, fluoride), azides, anhydrides or carbodiimides. In addition, reactive intermediates formed may be esters such as pentafluorophenyl esters or hydroxysuccinimido esters. Intermediates derived from acyl chlorides or azides are highly reactive. However, harsh reaction conditions and high reactivity are frequently a barrier to use for sensitive substrates or amino acids. Accordingly, amide coupling strategies that use carbodiimides such as DCC (dicyclohexylcarbodiimide) or DIC (diisopropylcarbodiimide) open up a broad spectrum of application. Frequently, especially in the case of solid-phase synthesis, additives are used in order to improve reaction efficiency. Aminium salts are highly efficient peptide coupling reagents with short reaction times and minimum racemization. With some additives, for example HOBt, it is even possible to completely prevent racemization. Aminium reagents are used in equimolar amounts with the carboxylic acid in order to prevent excessive reaction with the free amine of the peptide. Phosphonium salts react with carboxylate, which generally requires two equivalents of a base, for example DIEA. A significant advantage of phosphonium salts over iminium reagents is that phosphonium does not react with the free amino group of the amine component. This enables couplings in an equimolar ratio of acid and amine, and helps to avoid intramolecular cyclization of linear peptides and excessive use of costly amine components.

An extensive compilation of reaction strategies and reagents for amide couplings can be found in the following review articles:
Analysis of Past and Present Synthetic Methodologies on Medicinal Chemistry: Where Have All the New Reactions Gone?; D. G. Brown, J. Boström; J. Med. Chem. 2016, 59, 4443-4458;
Peptide Coupling Reagents, More than a Letter Soup; A. EI-Faham, F. Albericio; Chem. Rev. 2011, 111, 6557-6602;
Rethinking amide bond synthesis; V. R. Pattabiraman, J. W. Bode; Nature, Vol. 480 (2011) 22/29;
Amide bond formation: beyond the myth of coupling reagents; E. Valeur, M. Bradley; Chem. Soc. Rev., 2009, 38, 606-631.

The meaning of some of the terms used in the context of the invention is elucidated below.

Theranostics: Diagnosis and therapy of cancers using nuclear-medical pharmaceuticals.

Tracer: Synthetically produced, radiolabeled substance which is used in a very small amount and is converted within the organism without affecting the metabolism.

Labeling precursor: Chemical compound containing a chelator or a functional group for labeling with a radioisotope.

Target: Biological target structure, in particular (membrane-bound) receptor, protein, enzyme or antibody in the living organism to which a targeting vector binds.

Targeting vector: Chemical group or radical that functions as a ligand, agonist, antagonist or inhibitor for a biological target (e.g. a protein, enzyme or receptor) and has a high binding affinity for that target.

FAPi: FAP inhibitor that binds to and inhibits or deactivates the fibroblast activation protein (FAP).

FAPs: FAP substrate that binds to the fibroblast activation protein (FAP) and preferably initiates endocytosis.

Radiopharmaceutical: Radiolabeled chemical compound or labeling precursor complexed with a radioisotope for nuclear-medical diagnosis or theranostics.

Tris linker: Structural unit, group or radical capable of mutual conjugation of three other chemical structural units.

Linker: Structural unit, group or radical that bonds two functional groups to one another in each case, such as an FAP inhibitor (FAPi), an FAP substrate (FAPs), a cytotoxin (CT), a labeling group (MG) and optionally a tris linker (TL).

Spacer: Structural unit that functions as spacer between two other chemical structural units and counteracts steric hindrance.

Cytotoxin (cytostatic): compound that attacks, damages and/or destroys cells and is used in chemotherapy to therapy cancer.

Chelator: Structural unit capable of stably complexing a metallic radioisotope.

Residue of a chelator: Chelator as part of a compound of the invention.

Targeted drug delivery system: Chemical compound comprising a cytotoxic active ingredient, a cleavable linker for release of the cytotoxic active ingredient and a targeting vector for accumulation in tumor tissue, and optionally a further linker or spacer and a chelator for labeling with a radioisotope.

Active ingredient conjugate: Compound comprising a cytotoxin, a biological targeting vector and a cleavable linker.

Dual active ingredient conjugate: Compound comprising a cytotoxin, a biological targeting vector, a chelator and linker.

Schemes 7-25 below show examples of inventive compounds:

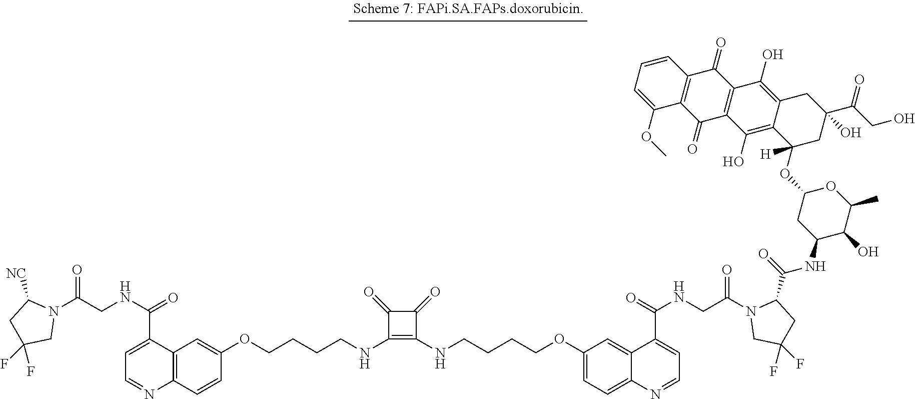 [SMILES: COc1cccc2c1C(=O)c1c(O)c3c(c(O)c1C2=O)C[C@@](O)(C(=O)CO)C[C@@H]3O[C@H]1C[C@H](NC(=O)[C@@H]2CC(F)(F)CN2C(=O)CNC(=O)c2ccnc3ccc(OCCCCNc4c(NCCCCOc5ccc6nccc(C(=O)NCC(=O)N7CC(F)(F)C[C@@H]7C#N)c6c5)c(=O)c4=O)cc23)[C@H](O)[C@H](C)O1]

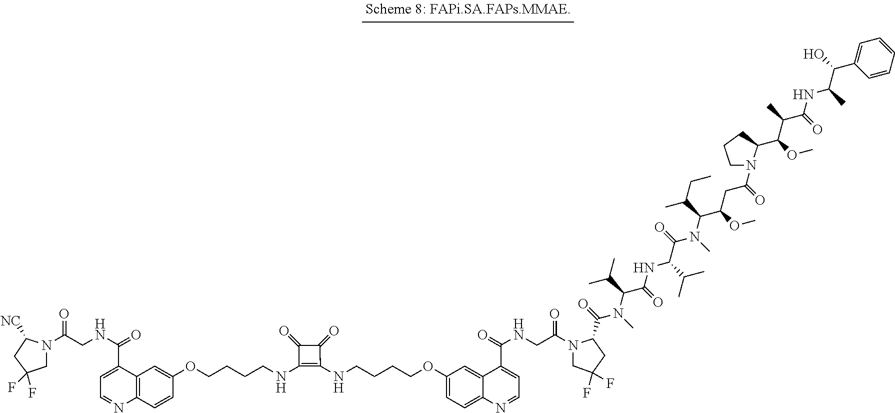 [SMILES: CCC(C)[C@@H]([C@@H](CC(=O)N1CCC[C@H]1[C@H](OC)[C@@H](C)C(=O)N[C@H](C)[C@H](O)c1ccccc1)OC)N(C)C(=O)[C@@H](NC(=O)[C@H](C(C)C)N(C)C(=O)[C@@H]1CC(F)(F)CN1C(=O)CNC(=O)c1ccnc2ccc(OCCCCNc3c(NCCCCOc4ccc5nccc(C(=O)NCC(=O)N6CC(F)(F)C[C@@H]6C#N)c5c4)c(=O)c3=O)cc12)C(C)C]

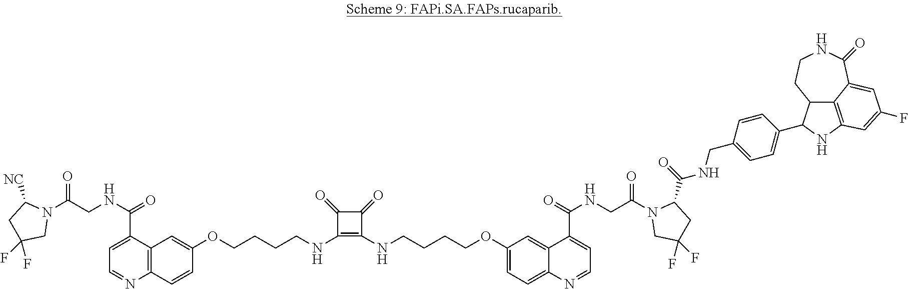 [SMILES: N#C[C@H]1CC(F)(F)CN1C(=O)CNC(=O)c1ccnc2ccc(OCCCCNc3c(NCCCCOc4ccc5nccc(C(=O)NCC(=O)N6CC(F)(F)C[C@H]6C(=O)NCc6ccc(C7Nc8cc(F)cc9c8C7CCNC9=O)cc6)c5c4)c(=O)c3=O)cc12]

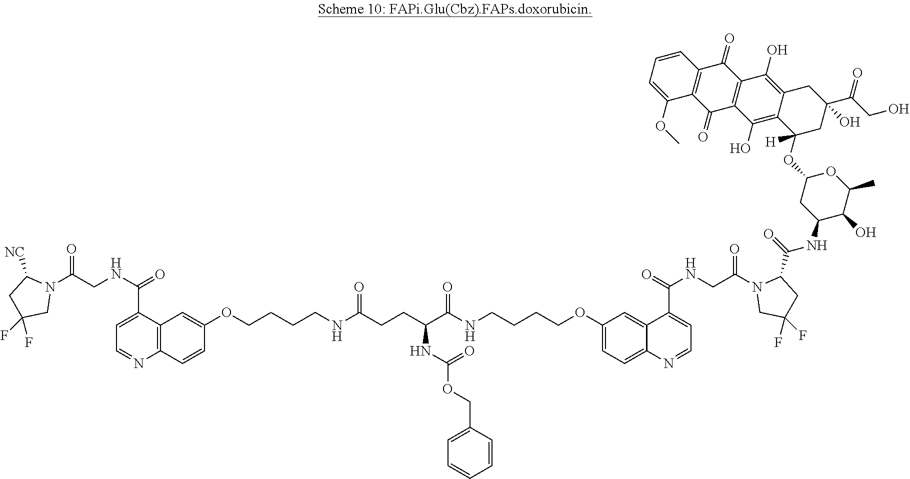 [SMILES: COc1cccc2c1C(=O)c1c(O)c3c(c(O)c1C2=O)C[C@@](O)(C(=O)CO)C[C@@H]3O[C@H]1C[C@H](NC(=O)[C@@H]2CC(F)(F)CN2C(=O)CNC(=O)c2ccnc3ccc(OCCCCNC(=O)[C@H](CCC(=O)NCCCCOc4ccc5nccc(C(=O)NCC(=O)N6CC(F)(F)C[C@@H]6C#N)c5c4)NC(=O)OCc4ccccc4)cc23)[C@H](O)[C@H](C)O1]

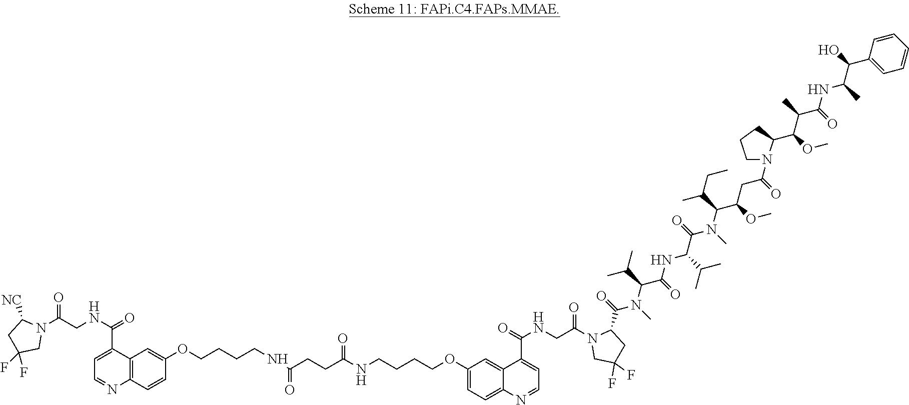 [SMILES: CCC(C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)[C@@H]1CC(F)(F)CN1C(=O)CNC(=O)c1ccnc2ccc(OCCCCNC(=O)CCC(=O)NCCCCOc3ccc4nccc(C(=O)NCC(=O)N5CC(F)(F)C[C@@H]5C#N)c4c3)cc12)C(C)C]

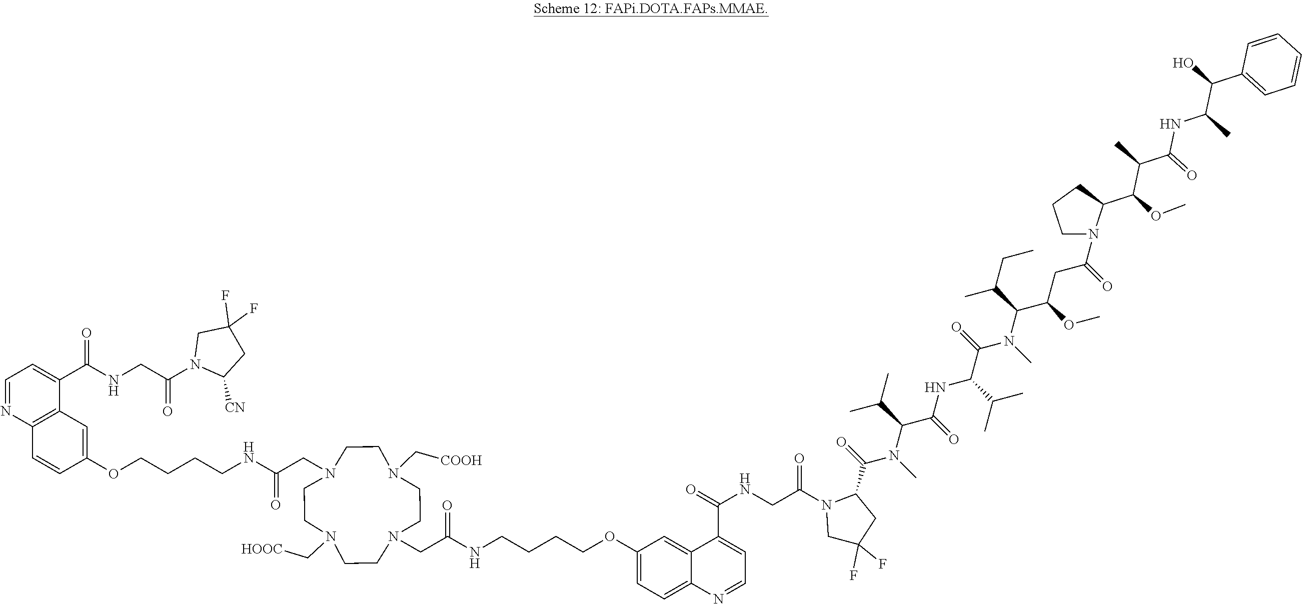 [SMILES: CCC(C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)[C@@H]1CC(F)(F)CN1C(=O)CNC(=O)c1ccnc2ccc(OCCCCNC(=O)CN3CCN(CC(=O)O)CCN(CC(=O)NCCCCOc4ccc5nccc(C(=O)NCC(=O)N6CC(F)(F)C[C@@H]6C#N)c5c4)CCN(CC(=O)O)CC3)cc12)C(C)C]

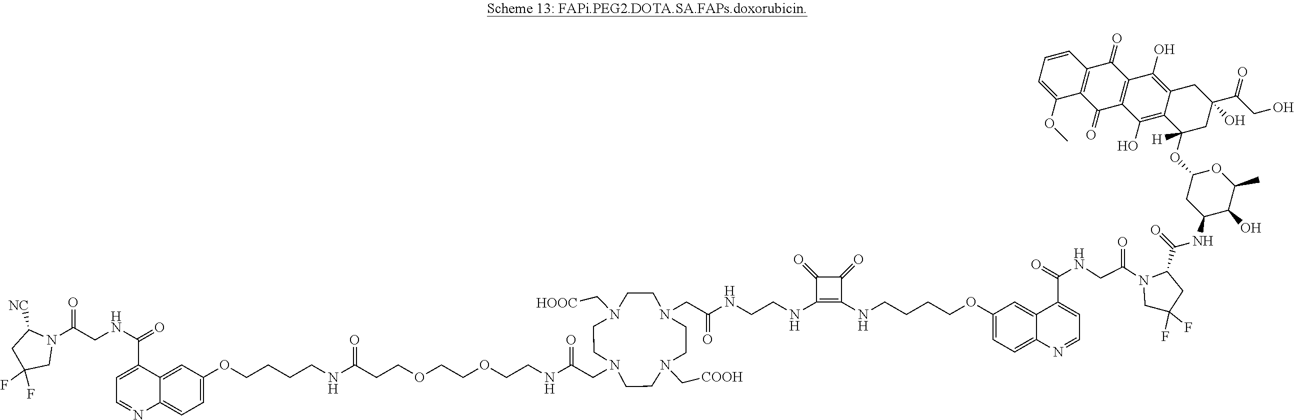 [SMILES: COc1cccc2c1C(=O)c1c(O)c3c(c(O)c1C2=O)C[C@@](O)(C(=O)CO)C[C@@H]3O[C@H]1C[C@H](NC(=O)[C@@H]2CC(F)(F)CN2C(=O)CNC(=O)c2ccnc3ccc(OCCCCNc4c(NCCNC(=O)CN5CCN(CC(=O)O)CCN(CC(=O)NCCOCCOCCC(=O)NCCCCOc6ccc7nccc(C(=O)NCC(=O)N8CC(F)(F)C[C@@H]8C#N)c7c6)CCN(CC(=O)O)CC5)c(=O)c4=O)cc23)[C@H](O)[C@H](C)O1]

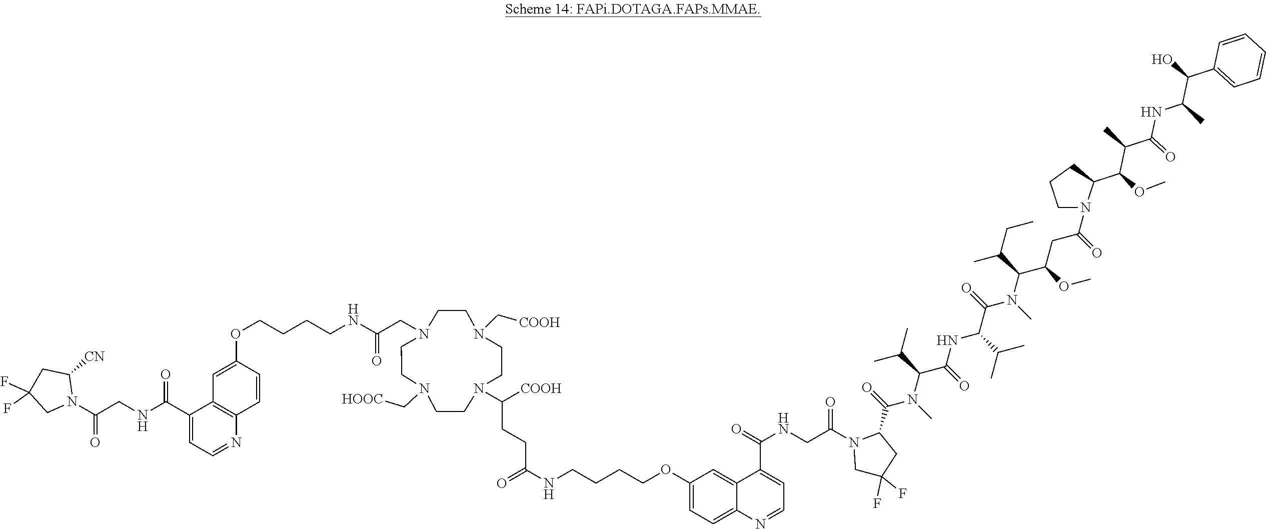 [SMILES: CCC(C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)[C@@H]1CC(F)(F)CN1C(=O)CNC(=O)c1ccnc2ccc(OCCCCNC(=O)CCC(C(=O)O)N3CCN(CC(=O)O)CCN(CC(=O)NCCCCOc4ccc5nccc(C(=O)NCC(=O)N6CC(F)(F)C[C@@H]6C#N)c5c4)CCN(CC(=O)O)CC3)cc12)C(C)C]

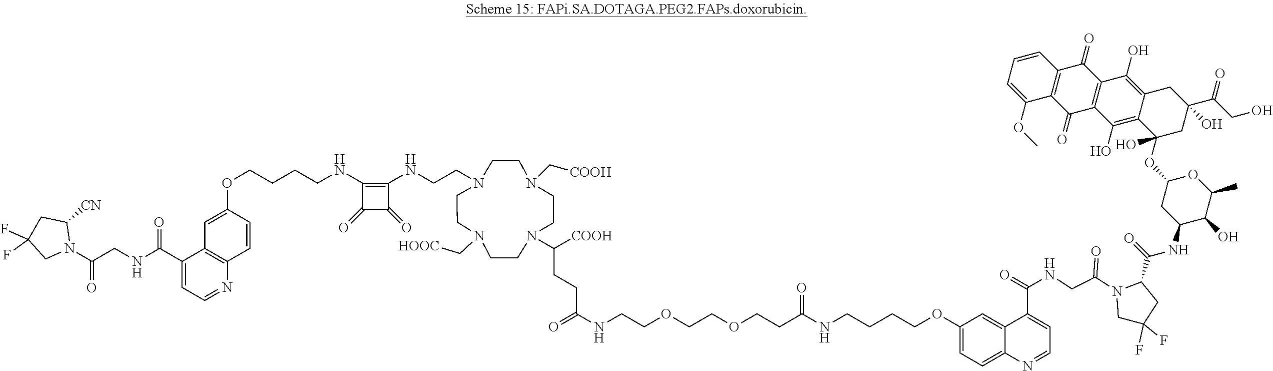 [SMILES: COc1cccc2c1C(=O)c1c(O)c3c(c(O)c1C2=O)C[C@@](O)(C(=O)CO)C[C@]3(O)O[C@H]1C[C@H](NC(=O)[C@@H]2CC(F)(F)CN2C(=O)CNC(=O)c2ccnc3ccc(OCCCCNC(=O)CCOCCOCCNC(=O)CCC(C(=O)O)N4CCN(CC(=O)O)CCN(CCNc5c(NCCCCOc6ccc7nccc(C(=O)NCC(=O)N8CC(F)(F)C[C@@H]8C#N)c7c6)c(=O)c5=O)CCN(CC(=O)O)CC4)cc23)[C@H](O)[C@H](C)O1]

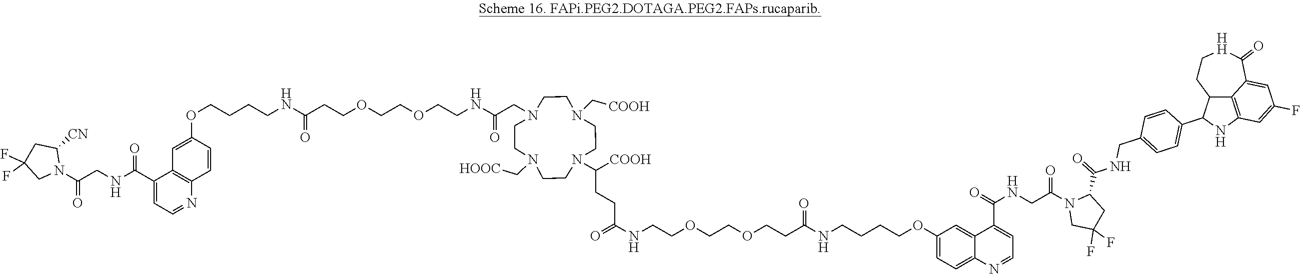

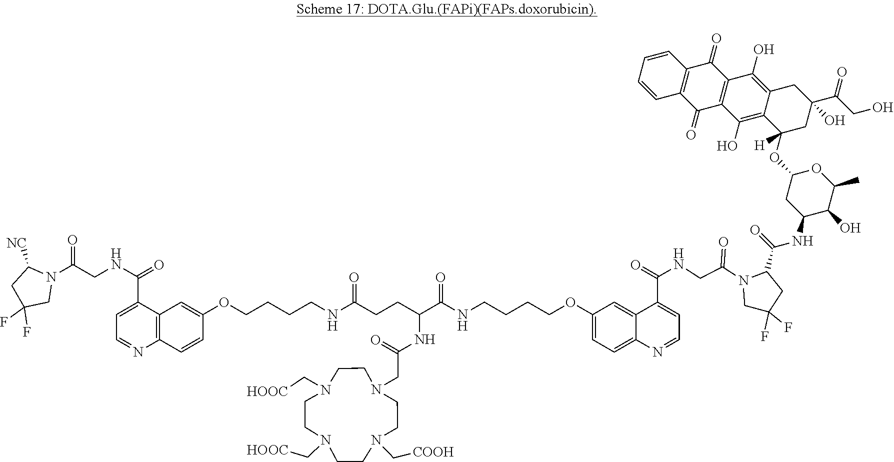

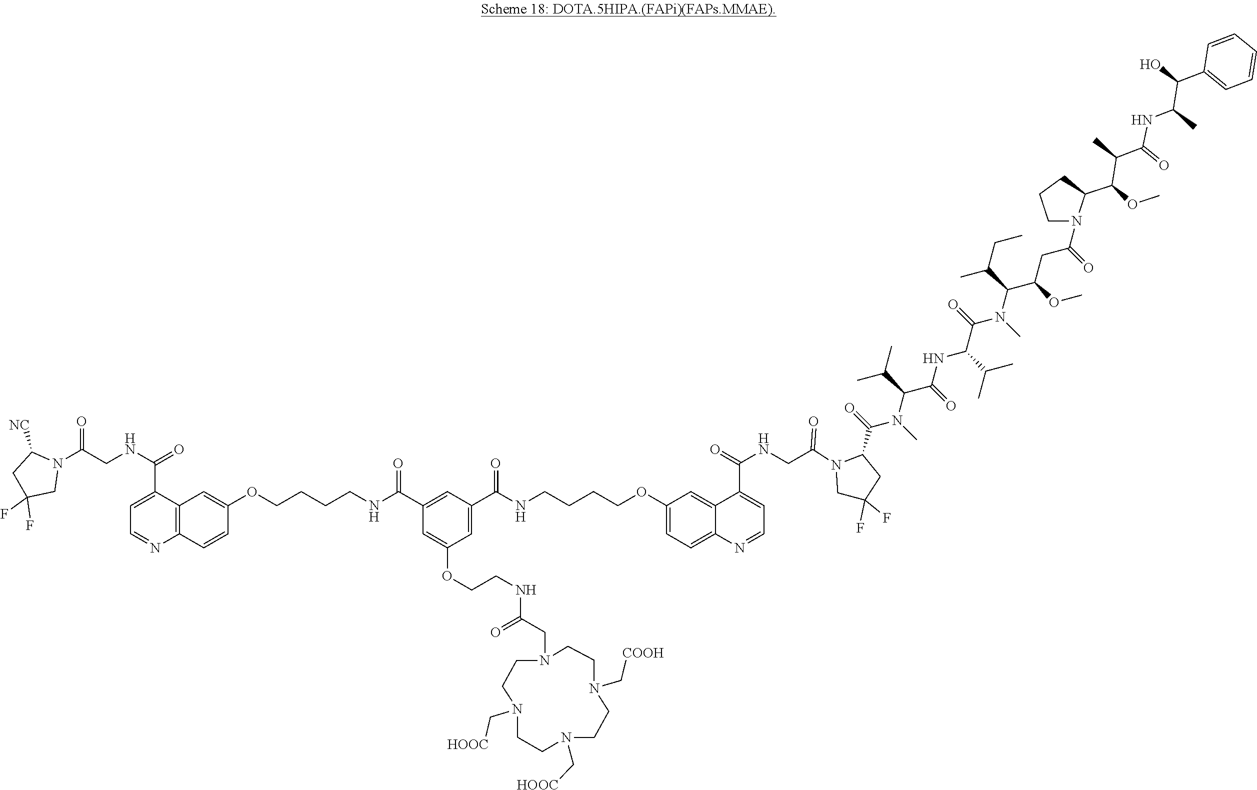 [SMILES: CCC(C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)[C@@H]1CC(F)(F)CN1C(=O)CNC(=O)c1ccnc2ccc(OCCCCNC(=O)c3cc(OCCNC(=O)CN4CCN(CC(=O)O)CCN(CC(=O)O)CCN(CC(=O)O)CC4)cc(C(=O)NCCCCOc4ccc5nccc(C(=O)NCC(=O)N6CC(F)(F)C[C@@H]6C#N)c5c4)c3)cc12)C(C)C]

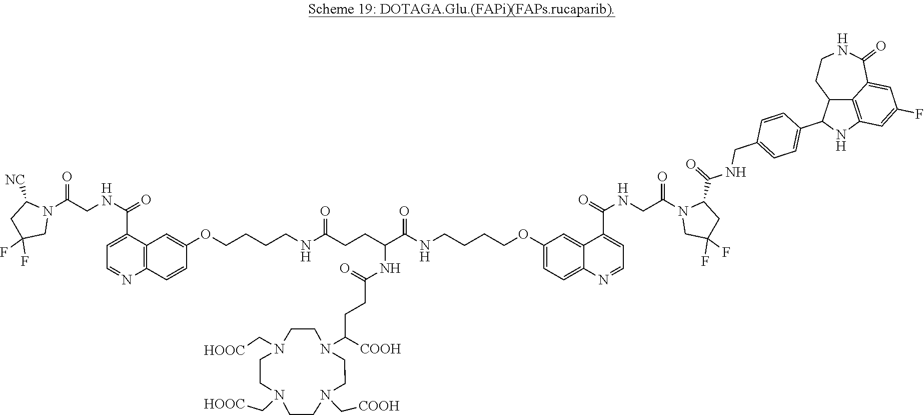 [SMILES: N#C[C@H]1CC(F)(F)CN1C(=O)CNC(=O)c1ccnc2ccc(OCCCCNC(=O)CCC(NC(=O)CCC(C(=O)O)N3CCN(CC(=O)O)CCN(CC(=O)O)CCN(CC(=O)O)CC3)C(=O)NCCCCOc3ccc4nccc(C(=O)NCC(=O)N5CC(F)(F)C[C@H]5C(=O)NCc5ccc(C6Nc7cc(F)cc8c7C6CCNC8=O)cc5)c4c3)cc12]

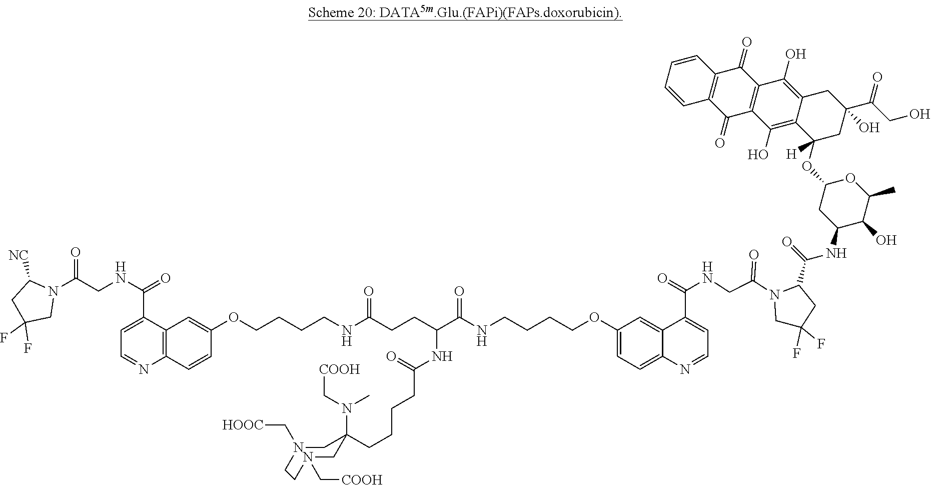

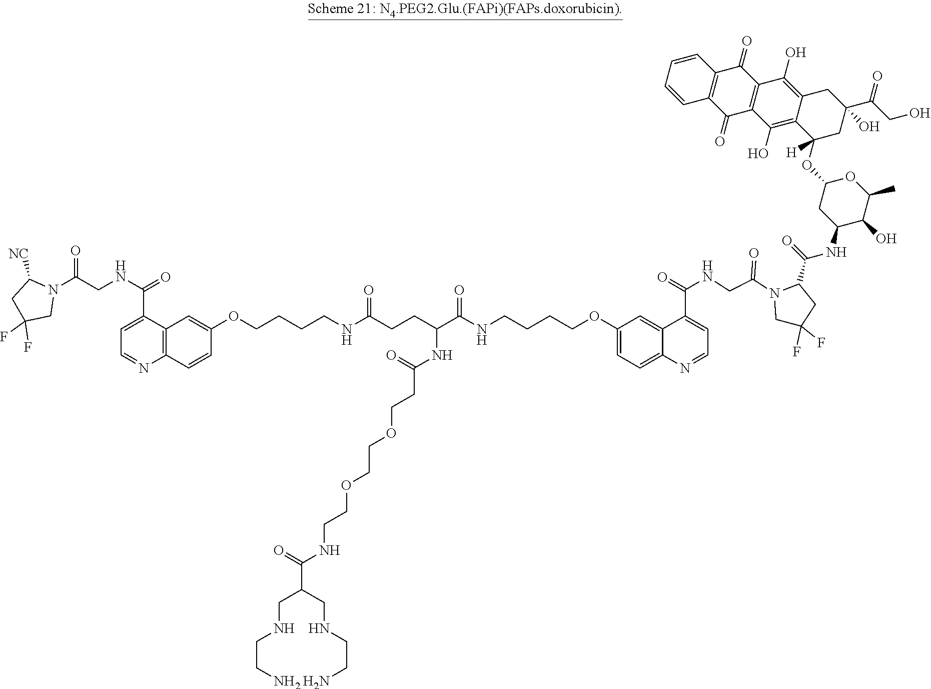

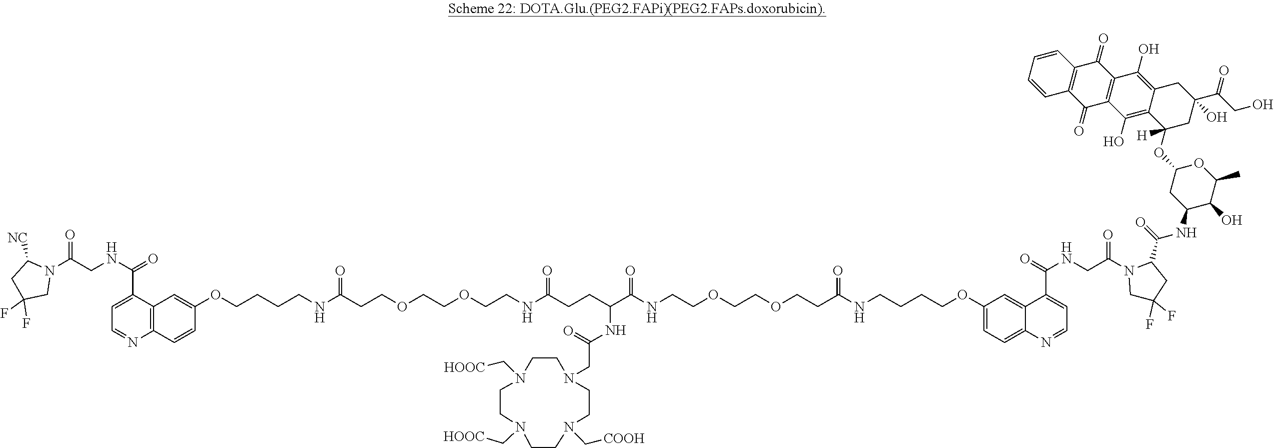

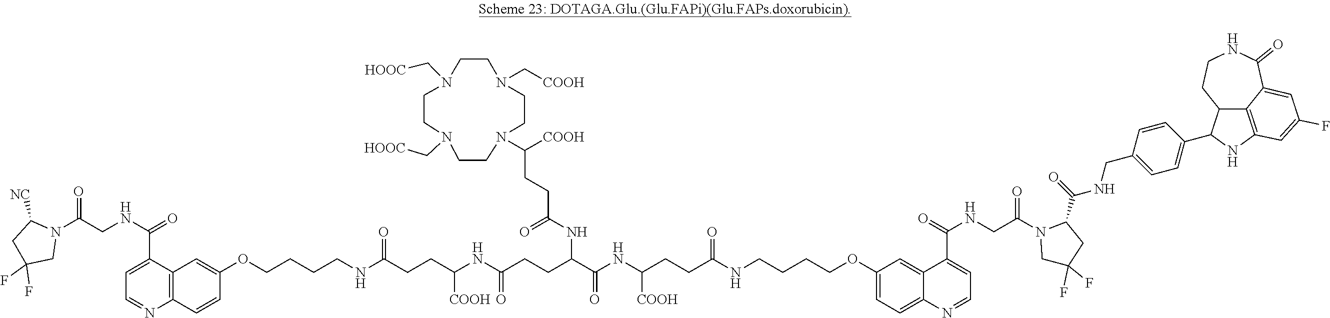 [SMILES: N#C[C@H]1CC(F)(F)CN1C(=O)CNC(=O)c1ccnc2ccc(OCCCCNC(=O)CCC(NC(=O)CCC(NC(=O)CCC(C(=O)O)N3CCN(CC(=O)O)CCN(CC(=O)O)CCN(CC(=O)O)CC3)C(=O)NC(CCC(=O)NCCCCOc3ccc4nccc(C(=O)NCC(=O)N5CC(F)(F)C[C@H]5C(=O)NCc5ccc(C6Nc7cc(F)cc8c7C6CCNC8=O)cc5)c4c3)C(=O)O)C(=O)O)cc12]

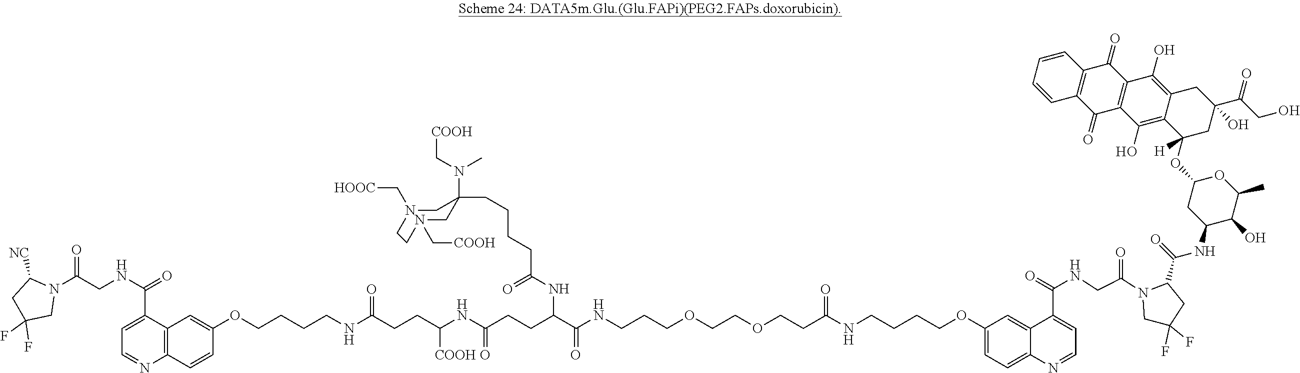

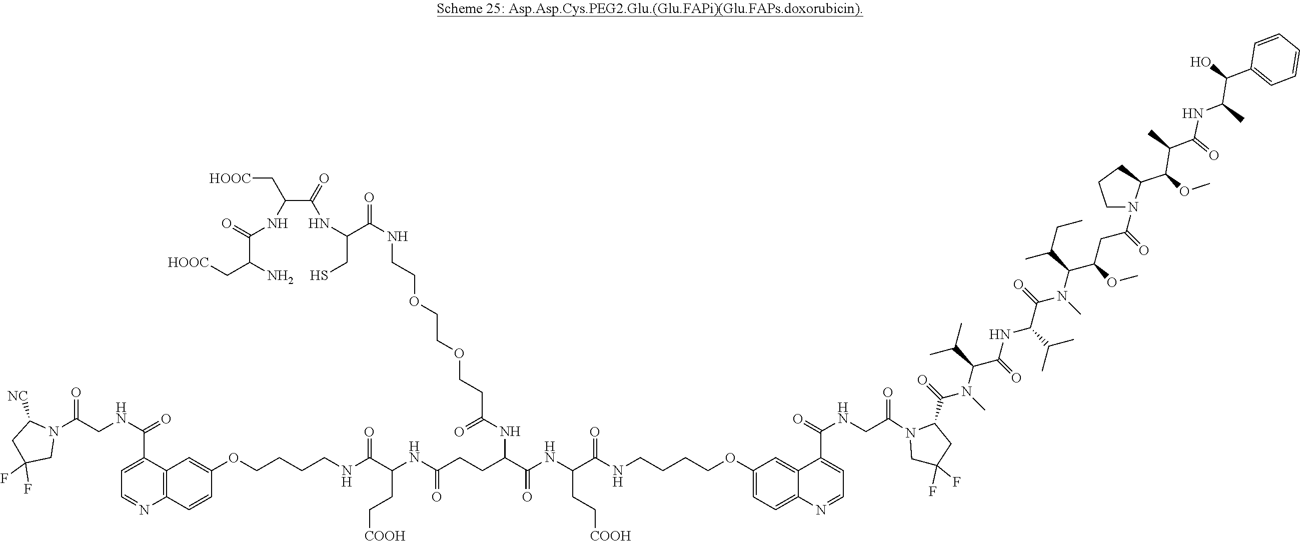 [SMILES: CCC(C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)[C@@H]1CC(F)(F)CN1C(=O)CNC(=O)c1ccnc2ccc(OCCCCNC(=O)C(CCC(=O)O)NC(=O)C(CCC(=O)NC(CCC(=O)O)C(=O)NCCCCOc3ccc4nccc(C(=O)NCC(=O)N5CC(F)(F)C[C@@H]5C#N)c4c3)NC(=O)CCOCCOCCNC(=O)C(CS)NC(=O)C(CC(=O)O)NC(=O)C(N)CC(=O)O)cc12)C(C)C]

1. A pharmaceutical compound for oncology applications having the structure 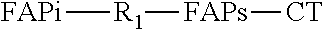
with 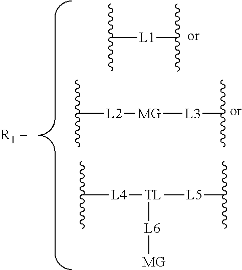
wherein CT denotes a cytotoxin, MG denotes a labeling group for a radioisotope, TL denotes a tris linker, and L1, L2, L3, L4, L5, L6 denote bivalent linkers;
FAPs is a substrate for the fibroblast activation protein (FAP) and has one of the structures [1] to [20]: 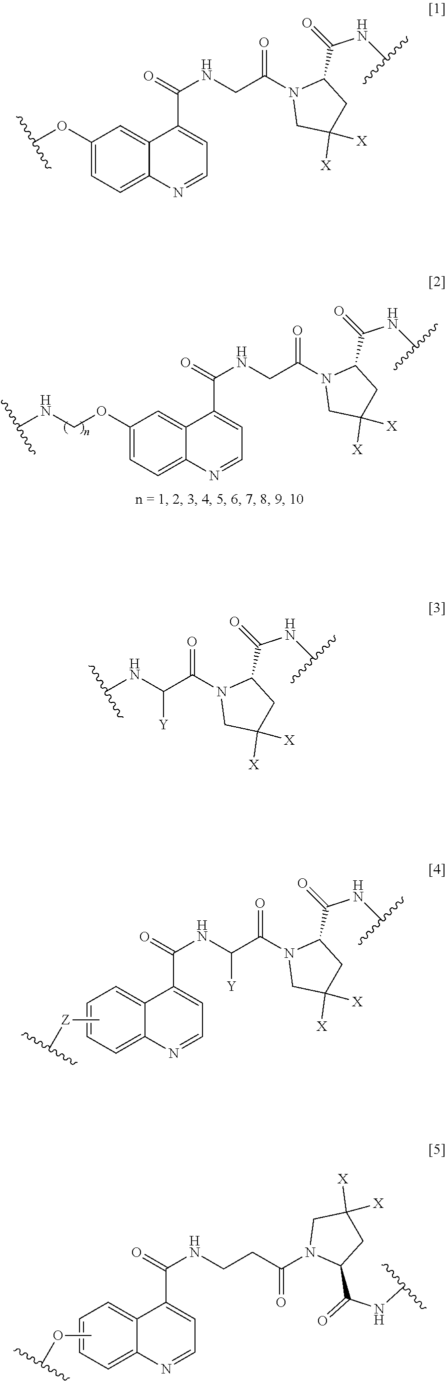 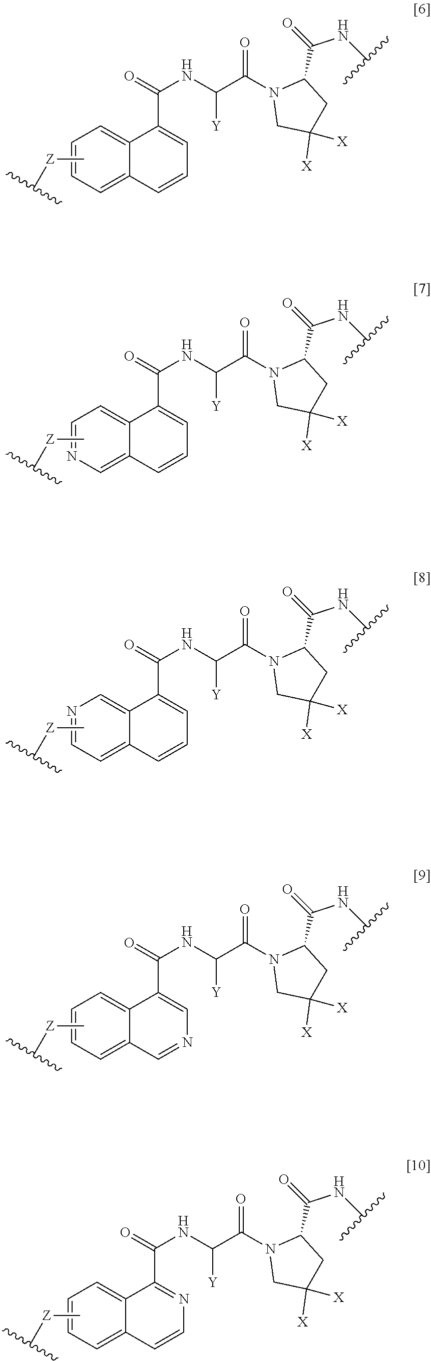 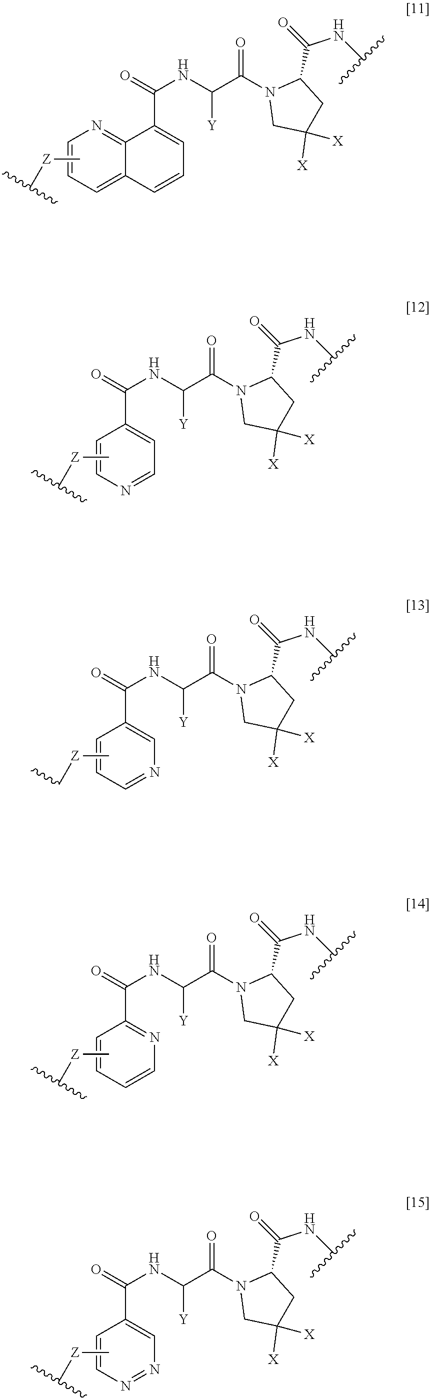 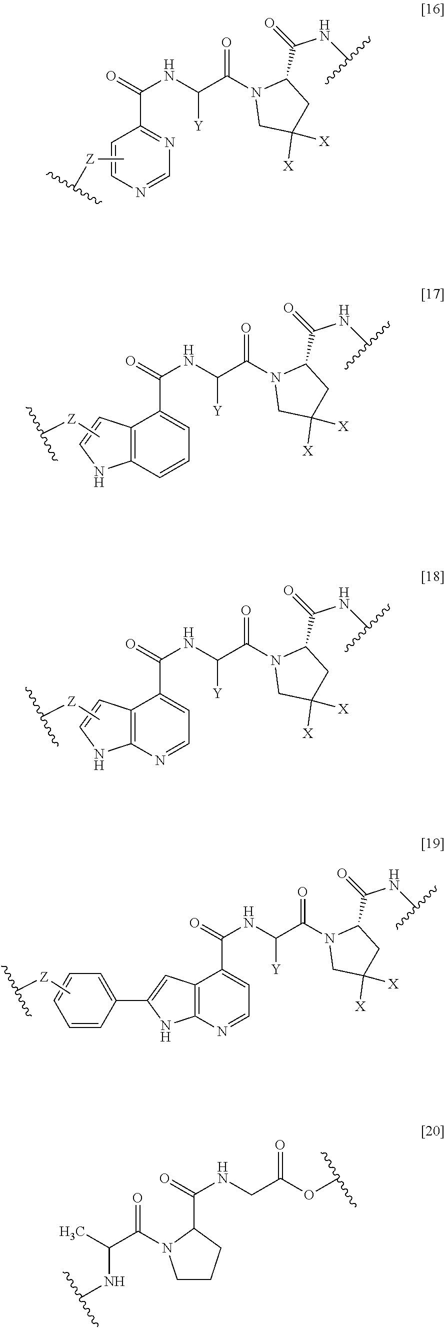
where [20] represents the amino acid sequence Ala-Pro-Gly;
where
X=H or F;
Y=H, CH₃, CH(CH₃)₂, C(CH₃)₃ or (CH₂),CH₃ with n=1, 2, 3, 4, 5, 6, 7, 8, 9 or 10; and
Z is chosen from alcohol, amidine, amine, amide, carboxamide, thioamide, imide, imide ester, imine, urea, thiourea, guanidine, carbonate, carboxylic ester, carbamate, ether, thioether, ester, ketone, phosphate, phosphonate, phosphinate, sulfonic ester, sulfinic ester, sulfone, thiol, disulfide, boronic ester, silyl ether radicals or derivatives thereof;
FAPi is an inhibitor of the fibroblast activation protein (FAP) and has one of the structures [21] to [46]: 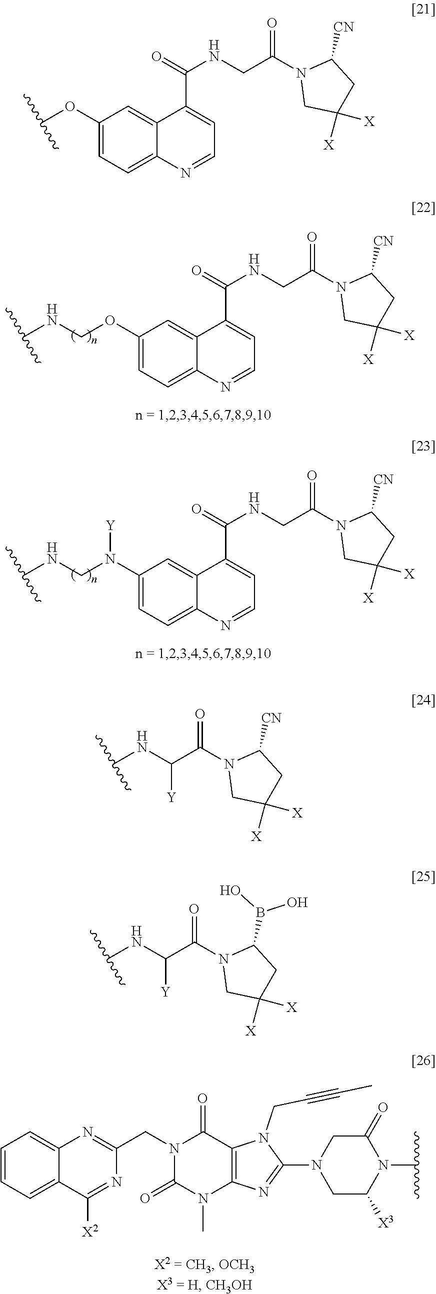 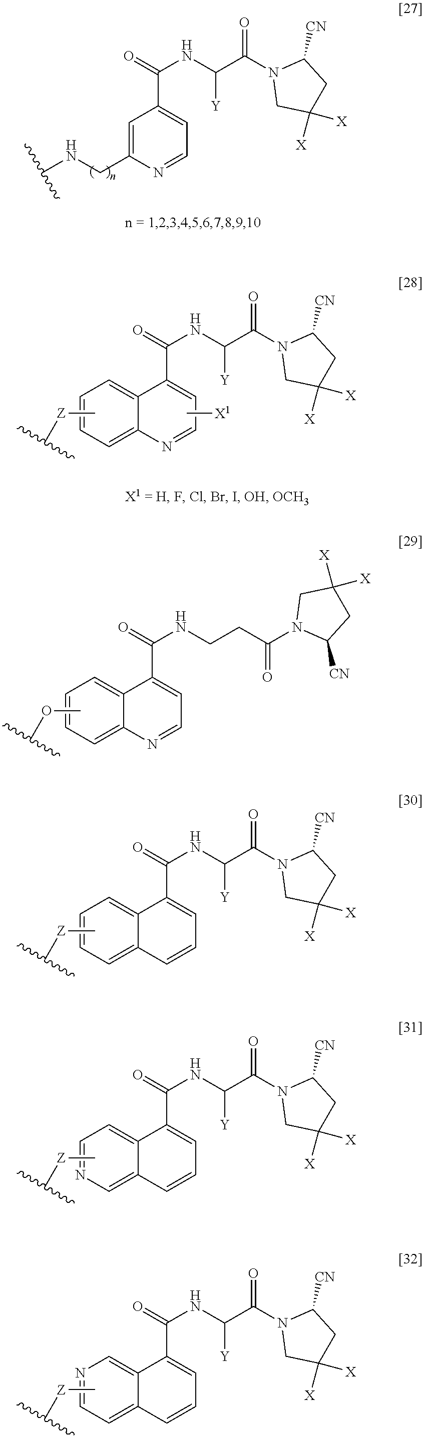 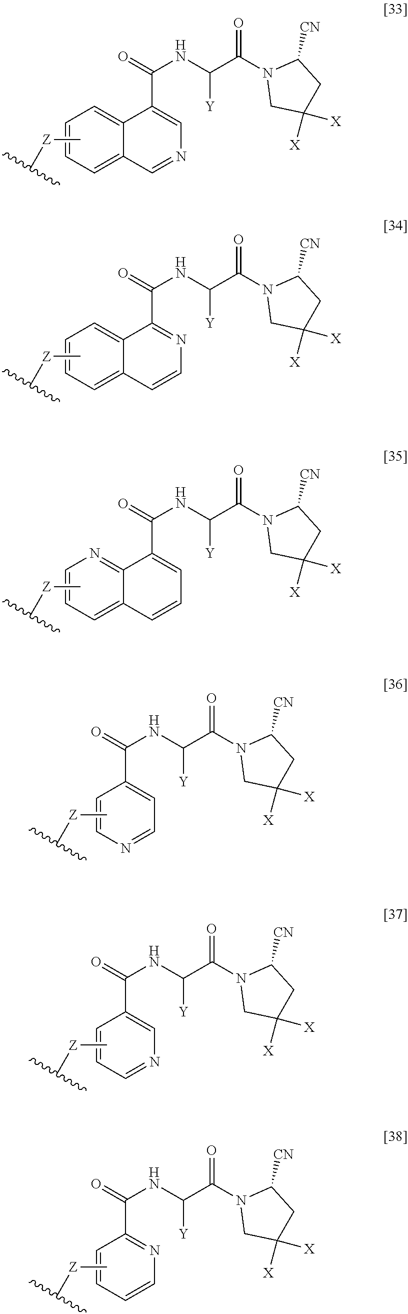 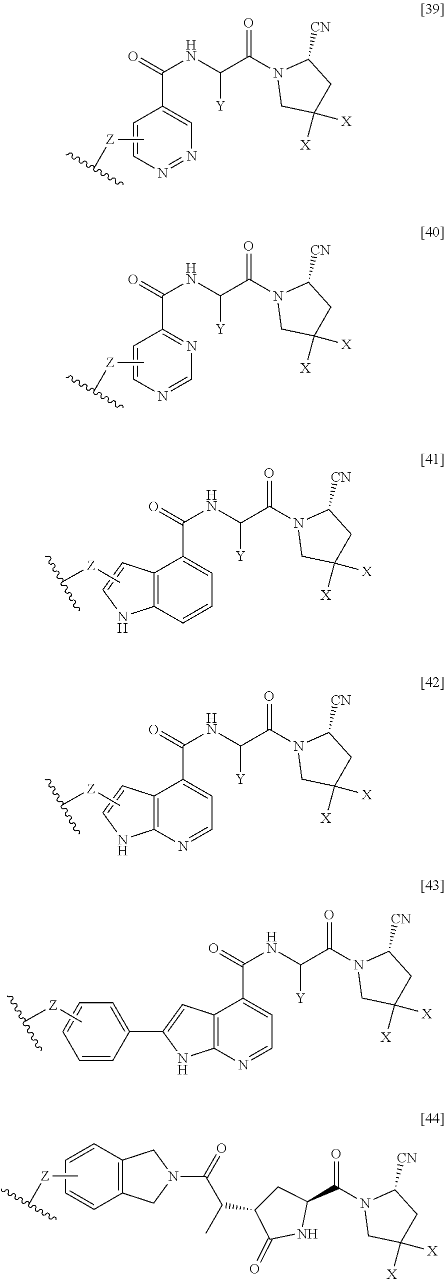 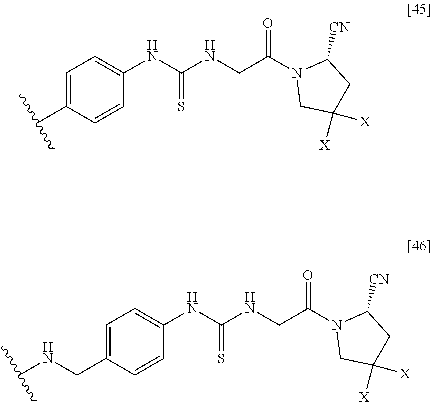
where
X=H or F;
Y=H, CH₃, CH(CH₃)₂, C(CH₃)₃ or (CH₂)ₙCH₃ with n=1, 2, 3, 4, 5, 6, 7, 8, 9 or 10;
Z is chosen from alcohol, amidine, amine, amide, carboxamide, thioamide, imide, imide ester, imine, urea, thiourea, guanidine, carbonate, carboxylic ester, carbamate, ether, thioether, ester, ketone, phosphate, phosphonate, phosphinate, sulfonic ester, sulfinic ester, sulfone, thiol, disulfide, boronic ester, silyl ether radicals or derivatives thereof.

2. The pharmaceutical compound as claimed in claim 1, wherein CT is a cytotoxin chosen from
antimetabolites;
alkylating cytotoxics;
mitosis inhibitors;
antibiotics;
enzyme inhibitors;
PARP inhibitors;
goserelin, leuprolide, metformin, NSC668394, tetrazole, α-solamargine, α-tomatine;
tubulin inhibitors;
tyrosine kinase inhibitors;
angiogenesis inhibitors;
hedgehog signaling pathway inhibitors;
VEGFR inhibitors; and
SERCA ATPase inhibitors.

3. The pharmaceutical compound as claimed in claim 1, wherein MG is a chelator chosen from the group comprising H₄pypa, ethylenediaminetetraacetate (EDTA), EDTMP diethylenetriamin-epenta(methylenephosphonic acid) (EDTMP), diethylenetriaminepentaacetate (DTPA) and derivatives thereof, nona-1,4,7-triaamine-triacetate (NOTA) and derivatives thereof, triazacyclononanephosphinic acid (TRAP), 1,4,7-triazacyclononane-1,4-bis[methylene(hydroxymethyl)phosphinic acid]-7-[methylene(2-carboxyethyl)phosphinic acid](NOPO), dodeca-1,4;7,10-tetraaminetetraacetate (DOTA), 2-(1,4,7,10-tetraazacyclododecane-4,7,10)pentanedioic acid (DOTAGA) and other DOTA derivatives, trideca-1,4,7,10-tetraaminetetraacetate (TRITA), tetradeca-1,4,8,11-tetraaminetetraacetate TETA) and derivatives thereof, pentadeca-1,4,7,10,13-pentaaminepentaacetate PEPA), (hexadeca-1,4,7,10,13,16-hexaaminehexaacetate (HEHA) and derivatives thereof, N,N′-bis(2-hydroxybenzyl)ethylenediamine-N,N′-diacetate (HBED) and derivatives thereof, DEDPA and derivatives thereof, deferoxamine (DFO) and derivatives thereof, trishydroxypyridinone (THP) and derivatives thereof, tetraazycyclodecanephosphinic acid (TEAP) and derivatives thereof, AAZTA 6-amino-6-methylperhydro-1,4-diazepane-N,N,N′,N′-tetraacetate AAZTA) and derivatives thereof 2,2′-(6-((carboxymethyl)(methyl)amino)-6-methyl-1,4-diazepane-1,4-diyl)diacetate (DATA) and derivatives thereof, 1-N-(4-aminobenzyl)-3,6,10,13,16,19-hexaazabicyclo[6.6.6]-eicosane-1,8-diamine (SAR) and derivatives thereof, 3-[(2′-amninoethyl)amino]-2-[(2″-amninoethyl) aminomethyl]propionic acid (N4) and other N₄ derivatives, 6-(4-isothiocyanatobenzyl)-3,3,9,9-tetramethyl-4,8-diazaundecane-2,10-dione dioxime (PnAO) and derivatives thereof, mercaptoacetyl-glycylglycine (MAG2) and derivatives thereof, mercaptoacetylglycylglycylglycine (MAG3) and derivatives thereof, mercaptoacetylserylserylserine (MAS3) and derivatives thereof, N-(2-mercaptoethyl)-2-[(2-mercaptoethyl)amino]acetamide MAIA) and derivatives thereof, ethylenedicysteine (EC) and derivatives thereof, dimercaptosuccinic acid (dmsa) and derivatives thereof, diaminodithiol (DADT), diaminodisulfide (DADS), N₂S₂ chelators and derivatives thereof, aminothiols and derivatives thereof; salts of the aforementioned chelators; hydrazinenicotinamide (HYNIC) and hydrazinenicotinamide derivatives.

4. The pharmaceutical compound as claimed in claim 1, wherein MG is dodeca-1,4,7,10-tetraaminetetraacetate (DOTA).

5. The pharmaceutical compound as claimed in claim 1, wherein MG is (1,4-bis(carboxymethyl)-6-[methylcarboxymethylamino]-6-pentanoic acid-1,4-diazepane (DATA^{5m}).

6. The pharmaceutical compound as claimed in claim 1, wherein MG is 1,4-bis(carboxymethyl)-6-[bis(carboxy-methyl)amino]-6-pentanoic acid-1,4-diazepane AAZTA).

7. The pharmaceutical compound as claimed in claim 1, wherein MG is a labeling group for the covalent binding of ¹⁸F, ¹³ or ²¹¹At.

8. The pharmaceutical compound as claimed in claim 7, wherein MG is chosen from

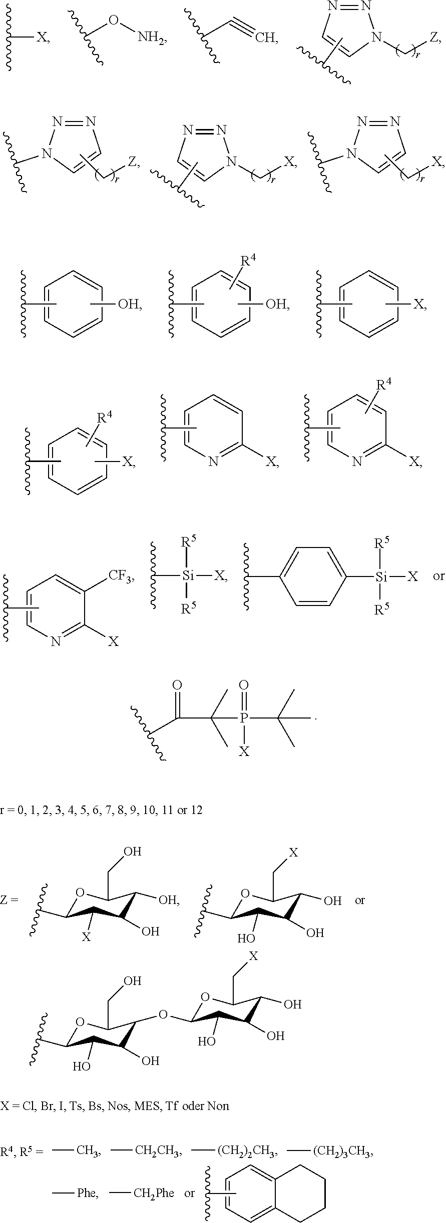

9. The pharmaceutical compound as claimed in claim 7, wherein MG is a group of the [symbol] -CF₂—X type with a leaving group X for substitution by ¹⁸F, ¹³¹I or ²¹¹At.

10. The pharmaceutical compound as claimed in claim 9, wherein MG contains a leaving group X chosen from a radical of bromine (Br), chlorine (Cl), iodine (I), tosyl (Ts), brosylate (Bs), nosylate (Nos), 2-(N-morpho-lino)ethanesulfonic acid (VIES), triflate (Tf) or nonaflate (Non).

11. The pharmaceutical compound as claimed in claim 1, wherein L1, L2, L3, L4, L5, L6 are independently chosen from 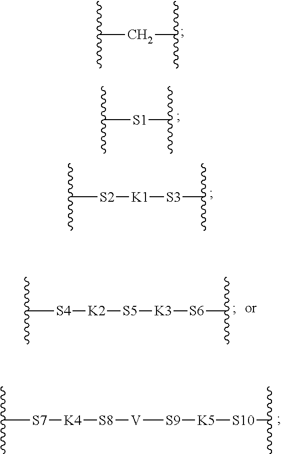
where S1, S2, S3, S4, S5, S6, S7, S8, S9, S10 are independently chosen from a mono- or oligomer composed of n alkyl units, polyethylene glycol (—(CH₂CH₂O)ₙ—), polypropylene glycol (—(CH₂CH(CH₃)₂O)ₙ—), polyalkylene glycol ether (—((CH₂)ₙO)—), polyamine (—((CH₂)ₙNH)—), polyamide (—((CH₂)ₙCONH)—), polyester (—((CH₂)ₙCOO)—), polyurethane (—((CH₂)ₙNHCOO)—), polyurea (—((CH₂)ₙNHCONH)—) or peptides having m amino acids with n=1, 2, 3, 4, 5, 6, 7, 8, 9, 10, 11, 12, 13, 14, 15, 16, 17, 18, 19 or 20;
where K1, K2, K3, K4, K5 are independently chosen from an alcohol, amidine, amine, amide, carboxamide, thioamide, imide, imide ester, imine, urea, thiourea, guanidine, carbonate, carboxylic ester, carbamate, ether, thioether, ester, ketone, phosphate, phosphonate, phosphinate, sulfonic ester, sulfinic ester, sulfone, thiol, disulfide, boronic ester, silyl ether radical or derivatives thereof; and
where V is chosen from
one of the structures [47], [48], [49] or [50]: 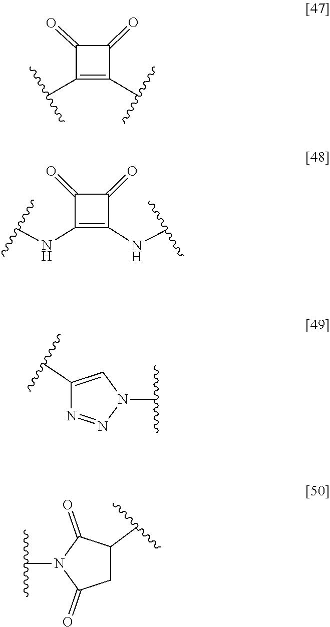 [SMILES: *c1c(*)c(=O)c1=O ; *Nc1c(N*)c(=O)c1=O ; *c1cn(*)nn1 ; *C1CC(=O)N(*)C1=O]
where [47] represents squaric acid, [48] squaric acid diamide, [49] a 1,4-disubstituted 1,2,3-triazole, and [50] a 1,3-disubstituted succinimidyl radical;
a residue of an amino acid; or
a benzene, phenol, cyclopentane, cyclohexane, pyridine, pyridazine, pyrimidine, pyrazine, piperidine, piperazine, 1,2,3-triazine, 1,2,4-triazine, 1,3,5-triazine, 1,2,3,4-tetrazine, 1,2,3,5-tetrazine, 1,2,4,5-tetrazine, thiazine, oxazine, pyrrole, pyrrolidine, pyrazole, imidazole, tetrahydroimidazole, 1,2,4-triazole, tetrazole, thiophene, furan, 1,2-thiazole, 1,3-thiazole, thiadiazole, 1,2-oxazole, 1,3-oxazole, 1,2,3-oxadiazole, 1,2,4-oxadiazole, 1,2,4-oxadiazole, 1,2,5-oxadiazole, naphthalene, indene, indole, isoindole, indazole, quinoline, isoquinoline, quinazoline, quinoxaline, cinnoline, phthalazine, tetrahydroquinoline, tetrahydroisoquinoline, quinolizidine, indolizidine, 2H-chromene, 4H-chromene, 2H-thiochromene, 4H-thiochromene, coumarin, purine radical or derivatives thereof.

12. The pharmaceutical compound as claimed in claim 1, wherein TL is chosen from one of the structures [51] to [115]:

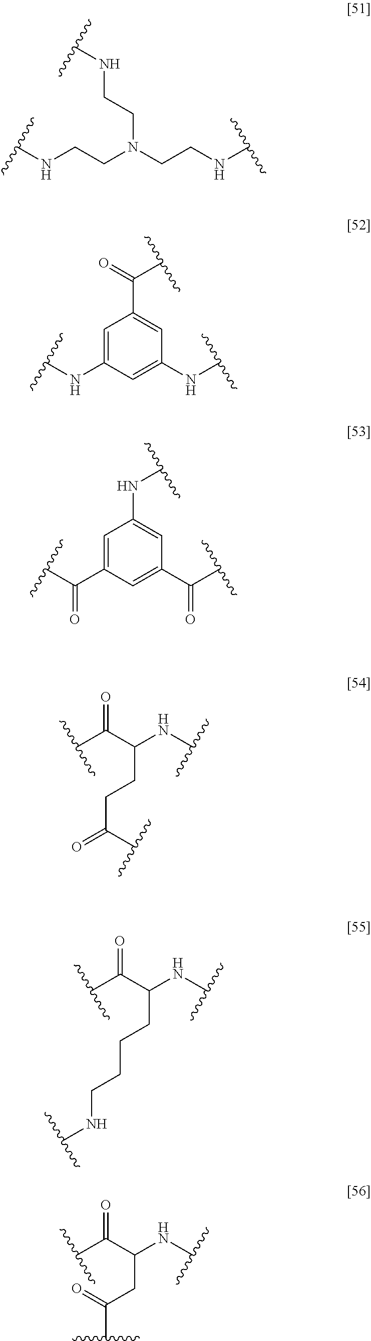 [SMILES: *NCCN(CCN*)CCN* ; *Nc1cc(N*)cc(C(*)=O)c1 ; *Nc1cc(C(*)=O)cc(C(*)=O)c1 ; *NC(CCC(*)=O)C(*)=O ; *NCCCCC(N*)C(*)=O ; *NC(CC(*)=O)C(*)=O]

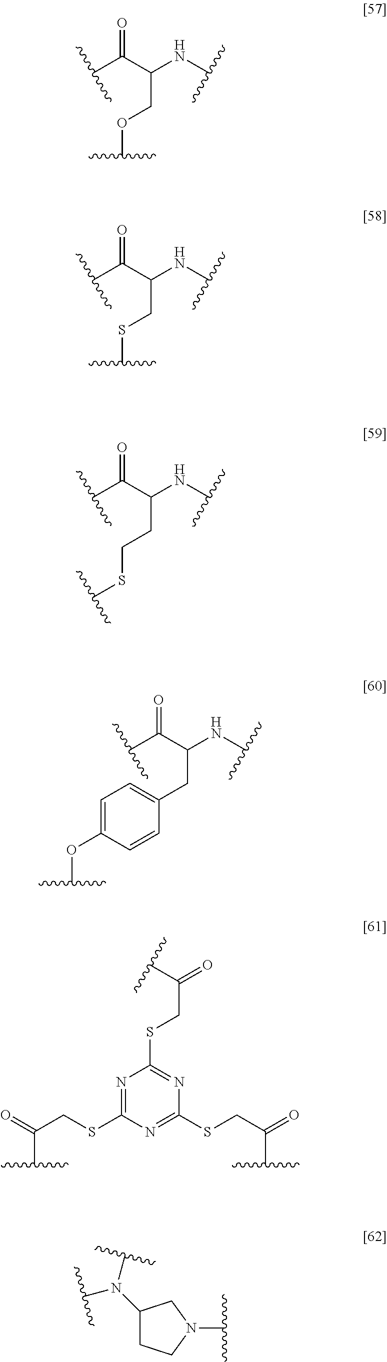 [SMILES: *NC(CO*)C(*)=O ; *NC(CS*)C(*)=O ; *NC(CCS*)C(*)=O ; *NC(Cc1ccc(O*)cc1)C(*)=O ; *C(=O)CSc1nc(SCC(*)=O)nc(SCC(*)=O)n1 ; *N1CCC(N(*)*)C1]

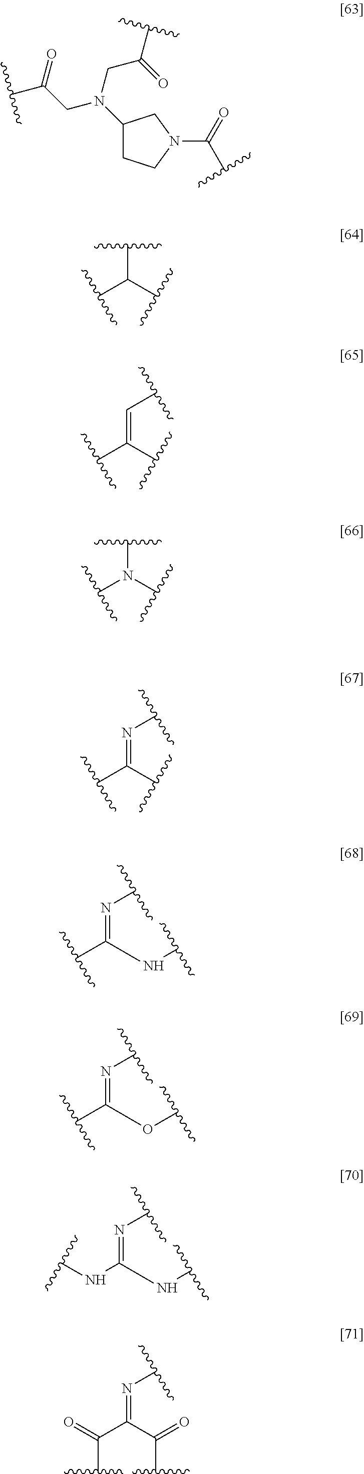

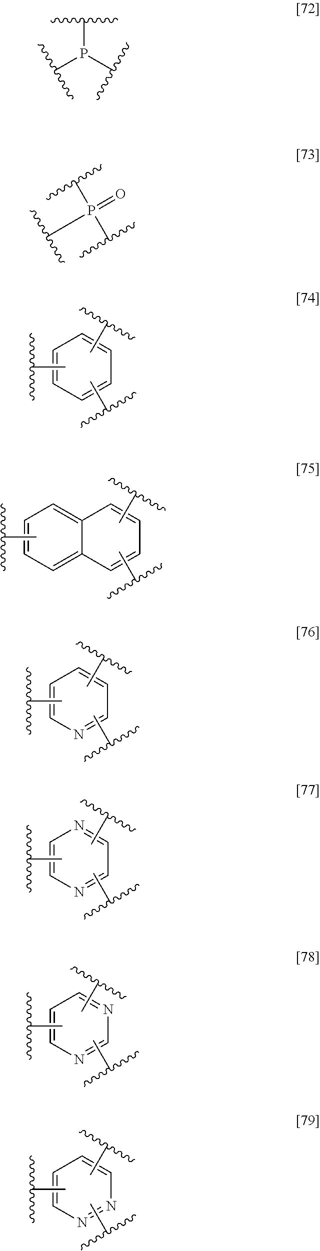

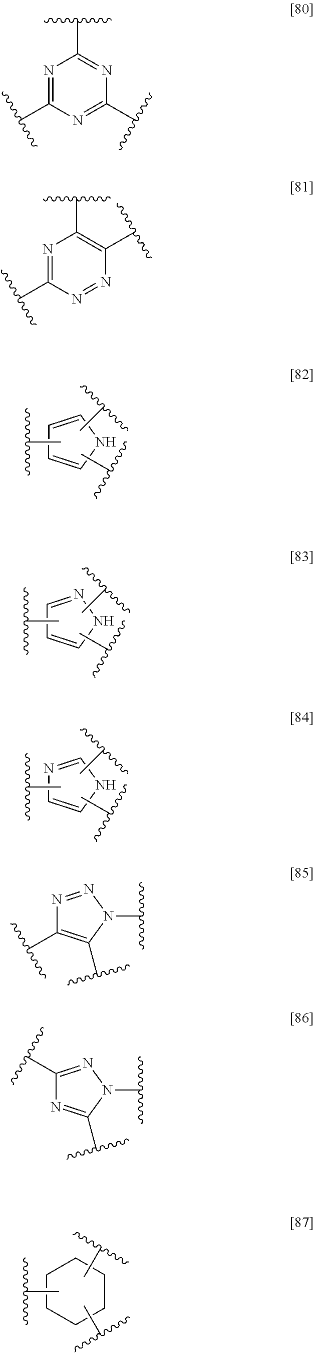

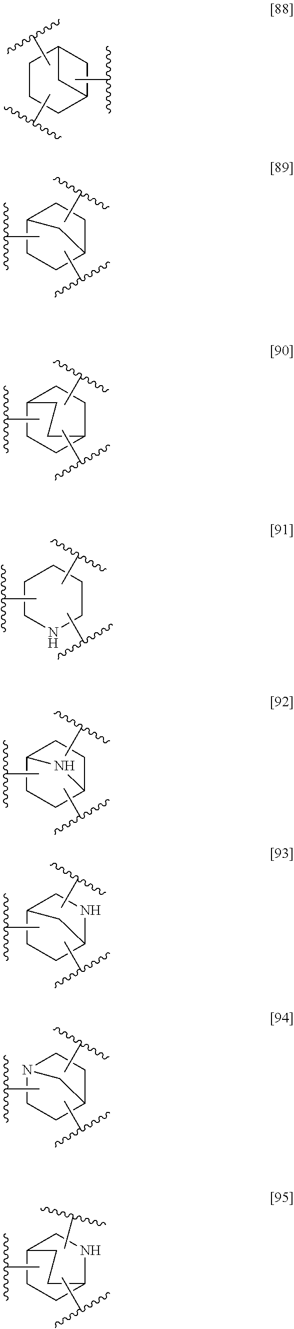

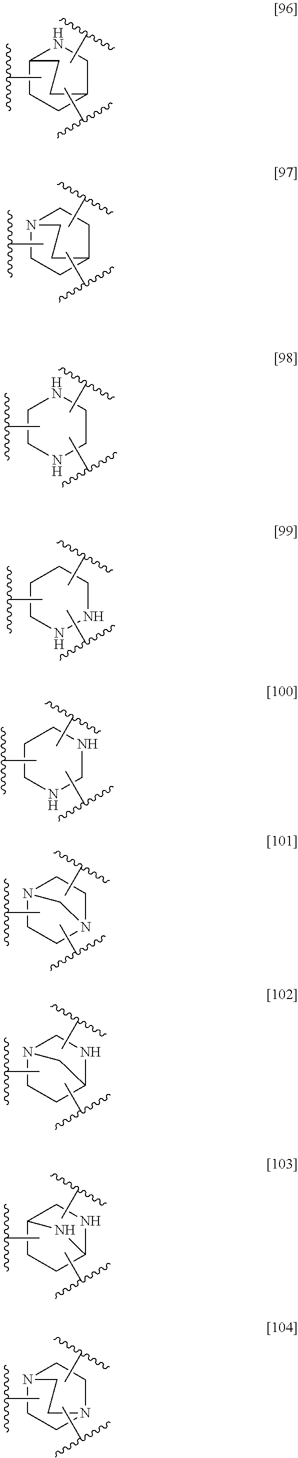

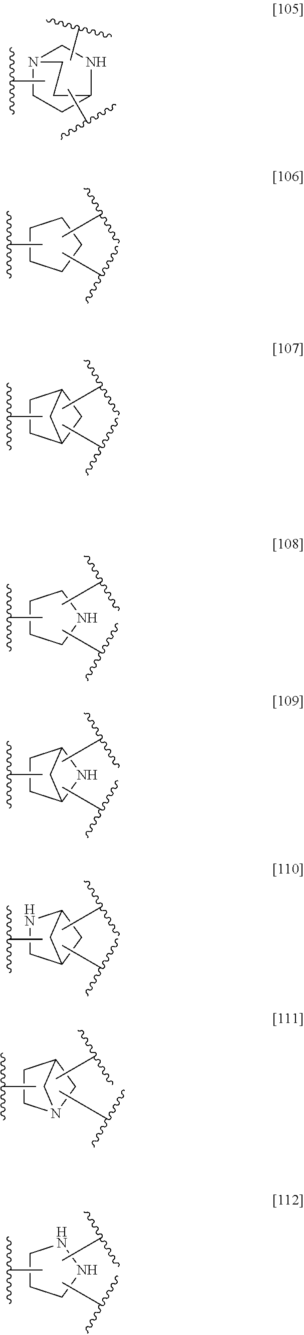

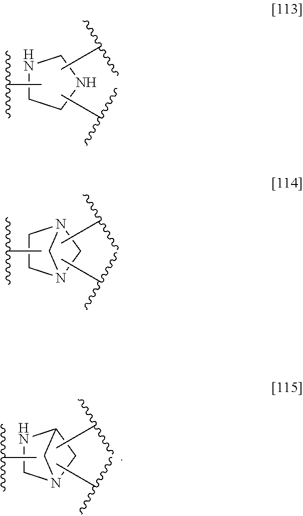

13. A radiotracer comprising a pharmaceutical compound as claimed in claim 1 and a radioisotope complexed therewith, said radioisotope chosen from ⁴⁴Sc, ⁴⁷Sc, ⁵⁵Co, ⁶²Cu, ⁶⁴Cu, ⁶⁷Cu, ⁶⁶Ga ⁶⁷Ga, ⁶⁸Ga ⁸⁹Zr, ⁸⁶Y, ⁹⁰Y, ⁸⁹Zr, ⁹⁰Nb, ^{99m}Tc, ¹¹¹In, ¹³⁵Sm, ¹⁴⁰Pr, ¹⁵⁹Gd, ¹⁴⁹Tb, ¹⁶⁰Tb, ¹⁶¹Tb, ¹⁶⁵Er, ¹⁶⁶Dy, ¹⁶⁶Ho, ¹⁷⁵Yb, ¹⁷⁷Lu, ¹⁸⁶Re, ¹⁸⁸Re, ²¹¹At, ²⁰³Pb, ²¹²Pb, ²¹³Bi, ²²⁵Ac or ²³²Th.

14. A radiotracer comprising a pharmaceutical compound as claimed claim 1 and a radioisotope conjugated therewith, said radioisotope chosen from ¹⁸F, ¹³¹I or ²¹¹At.

15. The pharmaceutical compound as claimed in claim 2, wherein
the antimetabolite is cytarabine, fludarabine, fluorouracil (5-FU), gemcitabine or methotrexate;
the alkylating cytotoxic is adozelesin, bizelesin, carzelesin, dacarbazine (DTIC), melphalan (BCNU), or temozolomide;
the mitosis inhibitor is monomethyl auristatin E (MMAE),
the antibiotic is dactinomycin, daunorubicin, doxorubicin, duocarmycin A, duocarmycin B1, duocarmycin B2, duocarmycin C1, duocarmycin C2, duocarmycin D, duocarmycin SA, idarubicin anthramycin, or mitoxantrone;
the enzyme inhibitor is L-asparaginase or motesanib;
the PARP inhibitor is rucaparib, olaparib, niraparib, veliparib, or iniparib;
the tubulin inhibitor is tubulysin B hydrazide;
the tyrosine kinase inhibitor is imatinib;
the angiogenesis inhibitor is Neovastat (AE-941);
the hedgehog signaling pathway inhibitor is as sonidegib;
the VEGFR inhibitor is sunitinib; and
the SERCA ATPase inhibitor is thapsigargin or a thapsigargin derivative selected from 12ADT, A12ADT or S12ADT.

16. The pharmaceutical compound as claimed in claim 3 wherein
the NOTA derivative is 1,4,7-triazacyclononane,1-glutaric acid,4,7-acetate (NODAGA), triazacyclononanephosphinic acid (TRAP) or 1,4,7-triazacyclononane-1,4-bis[methylene(hydroxymethyl)phosphinic acid]-7-[methylene(2-carboxyethyl)phosphinic acid](NOPO);
the HBED derivative is N,N′-bis[2-hydroxy-5-carboxyethyl)benzyl)ethylenediamine-N,N′-diacetate (HBED-CC);
the DEDPA derivative is 1,2-[[6-(carboxyl)pyridin-2-yl]methylamine]ethane (H₂dedpa) or 1,2-[[6-(carboxyl)pyridin-2-yl]methylamine]ethane-N,N′-diacetate (H₄octapa);
the TUP derivative is H₃THP-Ac or H₃TIP-mal (YM103);
the AAZTA derivative is 5-[(6-amino)-1,4-diazepane]pentanoic acid-N,N,N′,N′-tetraacetate (AAZTA⁵);
the DATA derivative is 5-[[6-(N-methyl)amino]-1,4-diacetate-1,4-diazepane]pentanoic acid-N,N′,N′-triacetate (DATA^{5m});
the SAR derivative is 1,8-diamino-3,6,10,13,16,19-hexaazabicyclo[6.6.6]eicosane ((NH₂)₂SAR);
the PnAO derivative is 3,3′-(1,4-butanediyldiamino)-bis(3-methyl-2-butanone) dioxime) (BMS181321); and
the MAG3 derivative is N₃S adipate.

17. The pharmaceutical compound as claimed in claim 11, wherein the amino acid residue is Ala, Arg, Asn, Asp, Cys, Gln, Glu, Gly, His, Ile, Leu, Lys, Met, Phe, Pro, Ser, Thr, Trp, Tyr, Val, Pyl, Sec, γ-aminobutyric acid (GABA), homoserine, 3,4-dihydroxy-phenylalanine (DOPA), citrulline, β-alanine or thyroxine.
